SEQUENCE LISTING (9 sequences)

SEQ ID NO: 1 - DNA, 30 bp, unidentified
  source 1..30: mol_type = genomic DNA
ccacaggctaaaaatgtatatgcccaaaga

SEQ ID NO: 2 - DNA, 3044 bp, unidentified
  source 1..3044: mol_type = genomic DNA
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaaatcgaacacaggctaaaaatgtatatgcccaaagataaaaaaaaaaaaaaaaaaaaaaaaaaaaaaaaaaaaaaaaaaaaaaaaaattcctgcagcccgggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 3 - DNA, 3087 bp, unidentified
  source 1..3087: mol_type = genomic DNA
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaaaaaaaaaaaaaaaaaaaaaaaaaaaaaaaaaaaaaaaaaaaaaaaaaaaccacaggctaaaaatgtatatgcccaaagataaaaaaaaaaaaaaaaaaaaaaaaaaaaaaaaaaaaaaaaaaaaaaaaattcctgcagcccgggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 4 - DNA, 125 bp, unidentified
  source 1..125: mol_type = genomic DNA
aaaaaaaaaaaaaaaaaaaaaaaaaaaaaaaaaaaaaaaaaaaaaaaaaagagcccactttaaaacaagcgcaggtaaaaaaaaaaaaaaaaaaaaaaaaaaaaaaaaaaaaaaaaaaaaaaaaa

SEQ ID NO: 5 - DNA, 76 bp, unidentified
  source 1..76: mol_type = genomic DNA
aaaaaaaaaaaaaaaaaaaaaaaaaaaaaaaaaaaaaaaaaaaaaaaaaagagcccactttaaaacaagcgcaggt

SEQ ID NO: 6 - DNA, 122 bp, unidentified
  source 1..122: mol_type = genomic DNA
aaaaaaaaaaaaaaaaaaaaaaaaaaaaaaaaaaaaaaaaaaaaaaaaaaatatggaaatacagcaccagcagaaaaaaaaaaaaaaaaaaaaaaaaaaaaaaaaaaaaaaaaaaaaaaaaa

SEQ ID NO: 7 - DNA, 73 bp, unidentified
  source 1..73: mol_type = genomic DNA
aaaaaaaaaaaaaaaaaaaaaaaaaaaaaaaaaaaaaaaaaaaaaaaaaaatatggaaatacagcaccagcag

SEQ ID NO: 8 - DNA, 80 bp, unidentified
  source 1..80: mol_type = genomic DNA
aaaaaaaaaaaaaaaaaaaaaaaaaaaaaaaaaaaaaaaaaaaaaaaaaacaccctgaagttgaaagtttggaggtgcca

SEQ ID NO: 9 - DNA, 130 bp, unidentified
  source 1..130: mol_type = genomic DNA
aaaaaaaaaaaaaaaaaaaaaaaaaaaaaaaaaaaaaaaaaaaaaaaaaacaccctgaagttgaaagtttggaggtgccaaaaaaaaaaaaaaaaaaaaaaaaaaaaaaaaaaaaaaaaaaaaaaaaaaa

(19) United States
(12) Patent Application Publication
(10) Pub. No.: US 2026/0071215 A1
(43) Pub. Date: Mar. 12, 2026

(54) SYSTEMS FOR ENHANCING TARGET MRNA EXPRESSION AND USES THEREOF

(71) Applicant: The Johns Hopkins University, Baltimore, MD (US)

(72) Inventors: Jeffery Coller, Frederick, MD (US); Bahareh Torkzaban, Jessup, MD (US)

(21) Appl. No.: 18/873,043

(22) PCT Filed: Jun. 9, 2023
(86) PCT No.: PCT/US2023/068224

§ 371 Date: Dec. 9, 2024

Related U.S. Application Data
(60) Provisional application No. 63/350,908, filed on Jun. 10, 2022.
(60) Provisional application No. 63/407,964, filed on Sep. 19, 2022.
(60) Provisional application No. 63/434,367, filed on Dec. 21, 2022.

(51) Int. Cl. C12N 15/113

(52) U.S. Cl. CPC C12N 15/113; C12N 2310/11

(57) ABSTRACT

Provided herein are nucleic acid agents that are or comprise one or more nucleic acid molecules, together comprising (a) a complementary element that hybridizes with a target mRNA; and (b) a poly(A) element. In some embodiments, the target mRNA is an mRNA of an active allele of a gene associated with a disorder associated with a decrease in the expression of a protein from the mRNA, wherein the disorder is a haploinsufficiency disorder.

### CROSS-REFERENCE TO RELATED APPLICATIONS

This application claims priority to U.S. Provisional Patent Application No. 63/350,908, filed on Jun. 10, 2022, U.S. Provisional Patent Application No. 63/407,964, filed on Sep. 19, 2022, and U.S. Provisional Patent Application No. 63/434,367, filed on Dec. 21, 2022, which are incorporated herein by reference in their entireties.

### SEQUENCE LISTING

This application contains a Sequence Listing that has been submitted electronically as an XML file named 44807-0434WO1_ST26.XML. The XML file, created on Jun. 7, 2023, is 15,501 bytes in size. The material in the XML file is hereby incorporated by reference in its entirety.

### BACKGROUND

Though changes in gene expression are commonly considered to reflect programmed transcriptional variability, it is a lesser-known fact that extensive regulation of messenger RNA expression also occurs during their translation. mRNA translation rate is a key feature defining post-transcriptional regulation. All transcripts are translated at unique rates and these rates can be controlled; dramatically impacting protein output per mRNA molecule. The cell achieves translational regulation through sequence and/or structural elements that recruit specific positive or negative acting factors to mRNAs.

Human mRNAs transmit genetic information from DNA to protein. Not only do mRNA transmit genetic information accurately, they also confer this information at the correct level. The amount of protein that comes from an mRNA can be determined by the translation rate of the mRNA, the time period over which translation occurs, and/or the stability of the mRNA transcript. And one key feature that times how long an mRNA will make its protein is its 3′ polyadenosine tail.

Nearly all human mRNAs bear a polyadenosine tail on their 3′ end. This tail has an average length of ˜200 nt in humans. The poly(A) tail serves as a master regulator of gene expression in the cytoplasm. Once an mRNA is in the cytoplasm, its poly(A) tail is subject to timed removal by a deadenylase enzyme complex. When the tail has been completely removed, translation of the mRNA stops and the mRNA is typically destroyed. Thus the poly(A) tail acts like a slow burning fuse, dictating how long a single mRNA will continue to make protein.

### SUMMARY

Targeting messenger RNA (mRNA) expression may offer a therapeutic method of modulating mRNA translation and/or treating haploinsufficiency disorders. Human mRNAs transmit genetic information from DNA to protein, wherein the amount of protein that comes from an mRNA can be determined by the translation rate of the mRNA, the time period over which translation occurs, and/or the stability of the mRNA transcript.

Described herein are molecular therapeutic strategies that use posttranscriptional regulation of a target mRNA to enhance protein expression from the target mRNA. A cell can achieve translational regulation through sequence and/or structural elements that recruit specific positive or negative acting factors to mRNAs, and one key feature that times how long an mRNA will make its protein is its 3′ polyadenosine tail. The poly(A) tail serves as a master regulator of gene expression in the cytoplasm, wherein once an mRNA is in the cytoplasm, its poly(A) tail is subject to timed removal by a deadenylase enzyme complex. When the tail has been completely removed, translation of the mRNA stops and the mRNA is typically destroyed. Thus the poly(A) tail acts like a slow burning fuse, dictating how long a single mRNA will continue to make protein.

Provided herein are nucleic acid agents comprising: (a) a complementary element that hybridizes with a target mRNA; and (b) a poly(A) region. In some embodiments, the complementary element hybridizes to a 3′ untranslated region of the target mRNA. In some embodiments, the complementary element comprises RNA and/or DNA.

In some embodiments, a provided nucleic acid agent is or comprises a single nucleic acid molecule that includes a complementary element (e.g., a complementary region) and a poly(A) element (e.g., a poly(A) region). In some such embodiments, the poly(A) region is located 5′ to the complementary region. In some embodiments, the poly(A) region is located 3′ to the complementary region. In some embodiments, the poly(A) region is located 3′ to the complementary region and the nucleic acid molecule further comprises a 5′ cap region located 5′ to the complementary region. In some embodiments, a nucleic acid molecule useful as a nucleic acid agent in accordance with the present disclosure comprises a first poly(A) region located 5′ to the complementary region and a second poly(A) region located 3′ to the complementary region.

In some embodiments, a poly(A) element (e.g., a poly(A) region) comprises about 20 nucleotides. In some embodiments, a poly(A) element (e.g., a poly(A) region) comprises about 50 nucleotides. In some embodiments, a poly(A) element (e.g., a poly(A) region) comprises about 75 nucleotides.

In some embodiments, a provided nucleic acid agent is or comprises a nucleic acid molecule that includes a nucleotide modification. In some embodiments, the nucleotide modification is a N1-methyladenosine (m1A), N6-methyladenosine (m6A), or adenosine to inosine (A-to-I) modification. In some embodiments, a relevant nucleotide modification is a pseudouracil, M1-pseudouracil, 5-methoxyuridine (5moU), or N4-acetylcytidine modification.

In some embodiments, a provided nucleic acid agent is or comprises a nucleic acid molecule is encoded by a sequence that comprises or consists of SEQ ID NO: 2 or SEQ ID NO: 3, or a sequence that has at least 80%, at least 85%, at least 90%, at least 95%, or at least 99% sequence identity to SEQ ID NO: 2 or SEQ ID NO: 3.

In some embodiments, a target mRNA is an mRNA that encodes a polypeptide of interest (e.g., a polypeptide whose increased expression provides an activity or benefit of interest, for example to or in a cell, tissue, system or organism in which it is expressed).

In some embodiments, a target mRNA is an mRNA of an active allele of a gene associated with a disorder associated with a decrease in the expression of a protein from the mRNA. In some embodiments, the disorder is a haploinsufficiency disorder. In some embodiments, the target mRNA encodes the MeCP2 protein. In some embodiments, the complementary region comprises or consists of SEQ ID NO: 1.

In some embodiments, a target mRNA encodes a reduced-activity variant of a polypeptide of interest.

In some embodiments, a target mRNA may be exogenous to a cell, tissue, organism or other system of interest. For example, in some embodiments, a target mRNA may be a therapeutic mRNA and/or may be a transcript of a gene included in or otherwise provided by a gene therapy or cell therapy agent.

In some particular embodiments, a target mRNA may encode a toxic or suicide polypeptide, for example whose expression reduces viability of (e.g., by inducing or promoting cell death apoptosis or cell death) a cell in which it is expressed or with which it otherwise comes into contact. In some particular such embodiments, such a cell may be a diseased cell (e.g., a cancer cell); alternatively or additionally, in some particular such embodiments, such a cell may be a cell therapy product (e.g., a preparation of engineered T cells such as a CAR-T cells, or of engineered NK cells, or of engineered B cells, etc).

Also provided herein are recombinant expression systems comprising a nucleic acid sequence encoding a nucleic acid that is or comprises one or both of (i) a complementary element (e.g., a complementary region) that hybridizes with a target mRNA and (ii) a poly(A) element (e.g., a poly(A) region). In some embodiments, the complementary region hybridizes to a 3′ untranslated region of a target mRNA as described herein. In some embodiments, the complementary element comprises RNA and/or DNA. In some embodiments, the poly(A) element is located 5′ to the complementary element. In some embodiments, the poly(A) element is located 3′ to the complementary element. In some embodiments, the poly(A) element is located 3′ to the complementary element and wherein the nucleic acid agent further comprises a 5′ cap element located 5′ to the complementary element. In some embodiments, the nucleic acid agent comprises a first poly(A) element located 5′ to the complementary element and a second poly(A) element located 3′ to the complementary element.

In some embodiments, the poly(A) element comprises about 20 nucleotides. In some embodiments, the poly(A) element comprises about 50 nucleotides. In some embodiments, the poly(A) element comprises about 75 nucleotides.

In some embodiments, the nucleic acid agent further comprises a nucleotide modification. In some embodiments, the nucleotide modification is a N1-methyladenosine (m1A), N6-methyladenosine (m6A), or adenosine to inosine (A-to-I) modification. In some embodiments, the nucleotide modification is a pseudouracil, M1-pseudouracil, 5-methoxyuridine (5moU), or N4-acetylcytidine modification.

In some embodiments, the recombinant expression system is encoded by a sequence that comprises or consists of SEQ ID NO: 2 or SEQ ID NO: 3, or a sequence that has at least 80%, at least 85%, at least 90%, at least 95%, or at least 99% sequence identity to SEQ ID NO: 2 or SEQ ID NO: 3.

In some embodiments, the target mRNA is an mRNA of an active allele of a gene associated with a disorder associated with a decrease in the expression of a protein from the mRNA. In some embodiments, the disorder is a haploinsufficiency disorder. In some embodiments, the target mRNA encodes the MeCP2 protein. In some embodiments, the nucleic acid molecule comprises or consists of SEQ ID NO: 1.

Also provided herein are expression vectors comprising any one of the recombinant expression systems described herein. In some embodiments, the expression vector is a viral vector. In some embodiments, the viral vector is an adeno-associated viral vector (AAV), a lentiviral vector, or an adenoviral vector.

Also provided herein are pharmaceutical compositions that comprise or deliver a nucleic acid agent as described herein (e.g., one or more nucleic acid molecules that together include (i) a complementary element; and/or (ii) a poly(A) element), a recombinant expression system as described herein (e.g., an expression vector as described herein).

Also provided herein are methods for increasing protein expression (e.g., increasing level of a protein (i.e., of a polypeptide)) encoded by an mRNA of interest as described herein.

In some embodiments, the present disclosure provides methods of treating a haploinsufficiency disorder in a subject, for example comprising administering to such subject a therapeutically effective amount of any of the pharmaceutical compositions described herein. In some embodiments, a therapeutically effective amount of the pharmaceutical composition enhances target mRNA protein expression. In some embodiments, a haploinsufficiency disorder is selected from the group consisting from 5qsyndrome, Adams-Oliver syndrome 1, Adams-Oliver syndrome 3, Adams-Oliver syndrome 5, Adams-Oliver syndrome 6, Alagille syndrome 1, Autoimmune lymphoproliferative syndrome type IA, Autoimmune lymphoproliferative syndrome type V, Autosomal dominant deafness-2A, Brain malformations with or without urinary tract defects (BRMUTD), Carney complex type 1, CHARGE syndrome, Cleidocranial dysplasia, Currarino syndrome, Denys-Drash syndrome/Frasier syndrome, Developmental delay, intellectual disability, obesity, and dysmorphic features (DIDOD), DiGeorge syndrome (TBXI-associated), Dravet syndrome, Duane-radial raysyndrome, Ehlers-Danlos syndrome (classic-like), Ehlers-Danlos syndrome (vascular type), Feingold syndrome 1, Frontotemporal lobar degeneration with TDP43 inclusions (FTLD-TDP), GRN-related, GLUT I deficiency syndrome, Greig cephalopolysyndactyly syndrome, Hereditary hemorrhagic telangiectasia type 1, Holoprosencephaly 3, Holoprosencephaly 4, Holoprosencephaly 5, Holt-Oram syndrome, Hypoparathyroidism, sensorineural deafness, andrenal disease (HDR), Kleefstra syndrome 1, Klippel-Trenaunay syndrome (AAGF-related), Leri-Weill dyschondrosteosis, Marfan syndrome, Mental retardation and distinctive facial features with or without cardiac defects (MRFACD), Mental retardation, autosomal dominant 1, Mental retardation, autosomal dominant 19, Mental retardation, autosomal dominant 29, Nail-patella syndrome (NPS), Phelan-McDermid syndrome, Pitt-Hopkins syndrome, Primary pulmonary hypertension 1, Rett syndrome (congenital variant), Smith-Magenis syndrome (RAII associated), Sotos syndrome 1, Sotos syndrome 2, Stickler syndrome type I, Supravalvular aorticstenosis, SYNGAPI-related intellectual disability, Treacher Collins syndrome, Trichorhinophalangeal syndrome type I, Ulnar-mammary syndrome, van der Woude syndrome1, Waardenburg syndrome type 1, Waardenburg syndrome type 2A, and Waardenburg syndrometype 4C. In some embodiments, the subject is a mammal. In some embodiments, the subject is a human.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-1C show exemplary schematics depicting design of an RNA Booster system (in red) as provided by the present disclosure. Three types of systems are shown, wherein each system has a region of homology to a gene of interest. This region is antisense to the mRNA of interest and will typically be antisense to a region in the 3′ untranslated region of the mRNA of interest. Each system has a polyadenosine tract (either 5′ of the antisense sequence (FIG. 1A), 3′ of the antisense sequence (FIG. 1B), or both (FIG. 1C)). Each system can bear a unique nucleotide modification at both the 5′ and 3′ end.

FIGS. 2A-2B show the results of western blotting directed against the MeCP2 protein. Cells were transfected with different RNA Booster systems bearing antisense regions to MeCP2 mRNA. GAPDH is shown as a control (FIG. 2A). Quantitation of relative protein expression is shown in FIG. 2B.

FIGS. 3A-3B show exemplary schematics depicting RNA Booster system (in red) designs. Two types of systems are shown, wherein each system has a region of homology to a gene of interest. Each system has a polyadenosine tract, either 5′ of the antisense sequence (FIG. 3A, upper panel) or 3′ of the antisense sequence and also including a 5′cap (FIG. 3A, lower panel). FIG. 3B shows results of western blotting directed against the MeCP2 protein. Cells were transfected with different RNA Booster systems bearing antisense regions to MeCP2 mRNA. GAPDH is shown as a control.

FIG. 4 shows results of MeCP2 expression in 3T3 cells incubated with Booster RNA for 16 hours, wherein results show both upcap short RNAs and capped RNAs.

FIG. 5 shows results of q-PCR for SYNGAP1 expression in SH-SY5Y cell incubated with SYNGAPI Booster RNA for 16 hours.

FIG. 6 shows results of qPCR in mouse liver indicating more than two fold increase of MeCP2 mRNA in the mice injected with Booster RNA.

FIG. 7 shows results of MeCP2 protein levels in mouse liver indicating up to two-fold increase of MeCP2 protein expression in the mice injected with Booster RNA. The Booster RNA was mixed with lipid nanoparticles (LNP) and injected into the tail vein. Each mouse was dosed with 25 μg Booster RNA.

FIG. 8 shows an exemplary schematic describing the design of an exemplary RNA Booster system. The depicted Booster RNA includes a region of homology to a gene of interest (e.g., antisense guide region). This region is antisense to the mRNA of interest and will typically be antisense to a region in the 3′ untranslated region of the mRNA of interest. The system also includes a polyadenosine tract (e.g., positioned at 5′ of the antisense sequence, and a unique nucleotide modification at the 5′ end.

FIGS. 9A-9B show results of qPCR assays, indicating an enhanced level of mRNA for both β-Catenin (FIG. 9A) and PurA (FIG. 9B) gene, 24 hours after transfection with booster RNA in Hek-293 cells.

FIGS. 10A-10B show results of deep brain perfusion of SYNGAPI booster RNAs in wild type animals, indicating significant alteration in the level of SYNGAPI in different brain regions, 24 hours post injection. Two different type of boosters (1 tail PA-SG, and 2 tails PA-SG-PA) targeting the same region on 3′UTR of SYNGAPI have been injected into the brain of the wild type mice. The result of both q-PCR (FIG. 10A) and Western Blotting (FIG. 10B) showed enhanced level of SYNGAPI in different brain regions. The results indicate 30 to 40% increase in the level of SYNGAPI in hippocampus, cortex and midbrain however there is no significant alteration in cerebellum.

FIG. 11 shows an exemplary schematic of SYNGAPI gene structure.

FIG. 12 shows results of targeting SYNGAPI compared with animals which were injected with RNA scramble and non-surgery animals, wherein deep brain perfusion was performed in more animals per condition. The results of RNA analysis and Western blotting indicated significant alteration in the level of SYNGAPI in the hippocampus, 48 hours post injection.

### DETAILED DESCRIPTION OF CERTAIN EMBODIMENTS

The present disclosure provides a discovery that targeting messenger RNA (mRNA) expression may offer a therapeutic method of modulating mRNA translation and/or treating haploinsufficiency disorders. mRNA translation rate is a key feature defining post-transcriptional regulation, where all transcripts are translated at unique rates and these rates can be controlled, dramatically impacting protein output per mRNA molecule. The cell can achieve translational regulation through sequence and/or structural elements that recruit specific positive or negative acting factors to mRNAs.

Described herein are molecular therapeutic strategies that use posttranscriptional regulation of a target mRNA to enhance protein expression from the target mRNA. Those skilled in the art, reading the present disclosure, will appreciate a variety of contexts in which provided technologies will be useful.

To give but a few examples, in some embodiments, provided systems may be designed and/or utilized to increase protein expression from a target mRNA that is endogenous to a host cell. Alternatively or additionally, in some embodiments, provided systems may be designed and/or utilized to increase protein expression from an mRNA that is heterologous to a host cell (e.g., that may have been introduced into such host cell or that may be progeny of—e.g., a transcription and/or replication product of a nucleic acid that has been introduced into such host cell). In some embodiments, provided systems and methods can provide for disease modifying treatment (e.g., haploinsufficiency disorders) by using a nucleic acid agent (e.g., “RNA booster”) that includes a key positive acting mRNA regulator (e.g., a polyA region) to bind and remain resident with the mRNA, thereby enhancing the wild-type (WT) mRNA's expression in a precise manner and restoring protein levels to normal.

In some embodiments, provided herein are nucleic acid agents that include (a) a complementary element that hybridizes with a target mRNA; and (b) a poly(A) element. In some embodiments, provided herein are nucleic acid agents that are or comprise nucleic acid molecules that include (a) a complementary region that hybridizes with a target mRNA; and (b) a poly(A) region.

Also provided herein are recombinant expression systems that include a nucleic acid sequence encoding a nucleic acid molecule comprising (i) a complementary element that hybridizes with a target mRNA and (ii) a poly(A) element.

Various non-limiting aspects of these systems are described herein, and can be used in any combination without limitation. Additional aspects of various components of these systems are known in the art.

### Certain Definitions

It must be noted that, as used in the specification and the appended claims, the singular forms “a,” “an” and “the” include plural referents unless the context clearly dictates otherwise.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete way(s). In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of, susceptibility to, severity of, stage of, etc the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “binding” typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

As used herein, a “cell” can refer to either a prokaryotic or eukaryotic cell, optionally obtained from a subject or a commercially available source.

As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). In some embodiments, an introduced polynucleotide may be stably maintained in the host cell; in some embodiments, an introduced polynucleotide may be transiently maintained. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery system. Examples of gene delivery systems can include, but are not limited to, lipid-based systems (e.g., liposomes, micelles, lipid nanoparticles, lipoplex), polymer-based systems (e.g., polymer particles (e.g., micro- or nano-particles)), metal-based systems (e.g., metal or metal-oxide particles (e.g., micro- or nano-particles)), or bacterial and/or viral-based, or viral-like, systems. In some embodiments, a delivery system may be or comprise one or more lipids, polypeptides, polysaccharides, metals, metal oxides, etc. Alternatively or additionally, in some embodiments, a delivery system may be or comprise one or more lipopolypeptides, lipopolysaccharides, liposomes, lipid nanoparticles, micelles, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, the term “designed” refers to an agent (i) whose structure is or was selected by the hand of man; (ii) that is produced by a process requiring the hand of man; and/or (iii) that is distinct from natural substances and other known agents.

As used herein, the term “encode” or “encoding” is used to describe the relationship between a nucleic acid and a polypeptide. For example, an mRNA whose sequence can be translated (e.g., by action of a ribosome) into a polypeptide is said to “encode” that polypeptide. Moreover, a nucleic acid (e.g., DNA or RNA) that, through one or more steps of replication (e.g., transcription, reverse transcription and/or other polymerization) and/or processing (e.g., splicing, capping, editing, etc) can be used to generate such an mRNA, is also said to “encode” the relevant polypeptide. A nucleic acid strand that encodes a polypeptide is referred to as a “coding” strand; its complement is an “antisense” strand.

As used herein, the term “exogenous” refers to a material introduced (e.g., by the hand of man) into a cell, a tissue or an organism that originates from a different source than the cell, tissue or organism into which it is being introduced, or otherwise is not naturally found in such cell, tissue or organism. Those skilled in the art will appreciate that such material may, in certain embodiments, still be considered to be “exogenous” to a cell, tissue or organism, that is a progeny cell, tissue or organism of that into which the material was originally introduced.

As used herein, the term “expression” refers to the process by which polynucleotides are transcribed (and/or optionally processed, such as by one or more of splicing, capping, editing, etc) into mRNA and/or the process by which an mRNA is translated into peptides, polypeptides, or proteins. In some embodiments, for example, if an expressed polynucleotide is or is derived from genomic DNA, expression may include splicing and/or other processing or modification to produce mRNA (e.g., in a eukaryotic cell). In some embodiments, expression level of a gene may be determined, for example, by measuring amount of mRNA, or protein encoded thereby, in a sample (e.g., a cell or tissue sample) in which the gene is expressed; in some embodiments expression level of multiple genes can be determined, e.g., substantially simultaneously, for example to establish an expression profile for a particular sample.

As used herein, the term “nucleobase” refers to the parts of nucleic acids that are involved in the hydrogen-bonding that binds one nucleic acid strand or sequence element to another complementary strand or sequence element in a sequence specific manner. The most common naturally-occurring nucleobases are adenine (A), guanine (G), uracil (U), cytosine (C), and thymine (T). In some embodiments, nucleobases are modified adenine, guanine, uracil, cytosine, or thymine. In some embodiments, nucleobases are methylated adenine, guanine, uracil, cytosine, or thymine. In some embodiments, a nucleobase comprises a heteroaryl ring wherein a ring atom is nitrogen, and when in a nucleoside, the nitrogen is bonded to a sugar moiety. In some embodiments, a nucleobase comprises a heterocyclic ring wherein a ring atom is nitrogen, and when in a nucleoside, the nitrogen is bonded to a sugar moiety. In some embodiments, a nucleobase is a “modified nucleobase,” e.g., a nucleobase other than adenine (A), guanine (G), uracil (U), cytosine (C), and thymine (T). In some embodiments, a modified nucleobase is substituted A, T, C, G or U. In some embodiments, a modified nucleobase is a substituted tautomer of A, T, C, G, or U. In some embodiments, a modified nucleobases is methylated adenine, guanine, uracil, cytosine, or thymine. In some embodiments, a modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. In some embodiments, as will be understood by those skilled in the art, the term “nucleobase” encompasses structural analogs used in lieu of natural or naturally-occurring nucleotides, such as modified nucleobases and nucleobase analogs. In some embodiments, a nucleobase is optionally substituted A, T, C, G, or U, or an optionally substituted tautomer of A, T, C, G, or U. In some embodiments, a “nucleobase” refers to a nucleobase unit in an oligonucleotide or a nucleic acid (e.g., A, T, C, G or U as in an oligonucleotide or a nucleic acid).

As used herein, the term “nucleoside” refers to a moiety wherein a nucleobase or a modified nucleobase is covalently bound to a sugar or a modified sugar. In some embodiments, a nucleoside is a natural nucleoside, e.g., adenosine, deoxyadenosine, guanosine, deoxyguanosine, thymidine, uridine, cytidine, or deoxycytidine. In some embodiments, a nucleoside is a modified nucleoside, e.g., a substituted natural nucleoside selected from adenosine, deoxyadenosine, guanosine, deoxyguanosine, thymidine, uridine, cytidine, and deoxycytidine. In some embodiments, a nucleoside is a modified nucleoside, e.g., a substituted tautomer of a natural nucleoside selected from adenosine, deoxyadenosine, guanosine, deoxyguanosine, thymidine, uridine, cytidine, and deoxycytidine. In some embodiments, a “nucleoside” refers to a nucleoside unit in an oligonucleotide or a nucleic acid.

As used herein, the term “nucleotide” refers to a monomeric unit of a polynucleotide that consists of a nucleobase, a sugar, and one or more internucleotidic linkages (e.g., phosphate linkages in natural DNA and RNA). The naturally occurring bases [guanine, (G), adenine, (A), cytosine, (C), thymine, (T), and uracil (U)] are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that, in various embodiments, as will be clear to those skilled in the art, naturally and non-naturally occurring sugar analogs are included. Nucleotides are linked via internucleotidic linkages to form nucleic acids, or polynucleotides. Various internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids. In some embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs. In some embodiments, a “nucleotide” refers to a nucleotide unit in a polynucleotide.

As used herein, “nucleic acid” in its broadest sense, refers to a compound and/or substance that is, or can be incorporated into, a polynucleotide chain. In some embodiments, a nucleic acid is a compound and/or substance that is, or can be incorporated into, a polynucleotide chain with a phosphodiester linkage. As will be clear from context, in some embodiments, “nucleic acid” refers to an individual nucleic acid residue (e.g., a nucleotide and/or nucleoside); in some embodiments, “nucleic acid” refers to a polynucleotide chain comprising individual nucleic acid residues. In some embodiments, a “nucleic acid” is or comprises ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), or a combination thereof. In some embodiments, a nucleic acid is, comprises, or consists of one or more natural nucleic acid residues. In some embodiments, a nucleic acid is, comprises, or consists of one or more nucleic acid residue analogs. In some embodiments, a nucleic acid analog differs from a nucleic acid in that one or more residues, and in some embodiments, are linked together other than by a phosphodiester. For example, in some embodiments, a nucleic acid includes one or more phosphorothioate and/or phosphoroamidite (e.g., 5′-N-phosphoramidite) linkages rather than phosphodiester bonds. In some embodiments, a nucleic acid includes one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), enzymatic synthesis in the absence of a complementary template, reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide.

Those skilled in the art will appreciate that various technologies, including, for example, site-directed mutagenesis, polymerase chain-reaction (PCR)-mediated mutagenesis, chemical mutagenesis, etc., can be utilized to introduce modifications into a nucleic acid.

Conservative amino acid substitutions are ones in which the amino acid residue is replaced with an amino acid residue having a similar side chain. Families of amino acid residues having similar side chains have been defined in the art. Exemplary such families amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

As used herein, the term “pharmaceutical composition” refers to a composition in which an active agent is formulated together with one or more pharmaceutically acceptable carriers. In some embodiments, a pharmaceutical composition is suitable for administration to a human or animal subject, e.g., via a particular route of administration (e.g., parenteral). In some embodiments, an active agent is present in a pharmaceutical composition in unit dose amount appropriate for administration in a therapeutic regimen that shows a statistically significant probability of achieving a predetermined therapeutic effect when administered to a relevant population.

As used herein, the term “polypeptide” generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

As used herein, “prevent” or “prevention” when used in connection with the occurrence of a disease, disorder, and/or condition, refers to reducing the risk of developing the disease, disorder and/or condition and/or to delaying onset of one or more characteristics or symptoms of the disease, disorder or condition. In some embodiments, prevention may be considered complete when onset of a disease, disorder or condition has been delayed for a predefined period of time.

As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

As used herein, the term “subject” refers an organism, typically a mammal (e.g., a human). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display a particular, or in some embodiments any, symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, the term “treatment” (also “treat” or “treating”) refers to administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. Thus, in some embodiments, treatment may be prophylactic; in some embodiments, treatment may be therapeutic.

As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### Provided Nucleic Acid Agents

The present disclosure provides certain nucleic acid agents as described herein, which may be useful, for example, in enhancing mRNA translation. In some embodiments, a provided nucleic acid agent is or comprises a nucleic acid molecule or molecules.

In some embodiments, a provided nucleic acid agent includes (a) a complementary element; linked or otherwise associated with (b) a poly(A) element. In some embodiments, a provided nucleic acid agent is comprised of two or more distinct nucleic acid molecules, associated with one another for example by hybridization (e.g., by way of complementary associating elements on different molecules, which associating elements hybridize with one another and thereby cause the separate nucleic acid molecules to associate with one another and form a provided nucleic acid agent, and/or by covalent association such as chemical linkage (e.g., via phosphodiester or other internucleoside linkage or by, for example, a bond created by chemical reaction such as via click chemistry). In many embodiments, a provided nucleic acid agent is comprised of a single nucleic acid molecule (e.g., strand), in which the complementary element and poly(A) element are distinct regions of the same strand.

Thus, in some embodiments, provided herein are nucleic acid molecules that include (a) a complementary region that hybridizes with a target mRNA; and (b) a poly(A) region.

Also provided herein are recombinant expression systems that include a nucleic acid sequence encoding one or both of a complementary element and a poly(A) element, as described herein. For example, in some embodiments, a provided recombinant expression system encodes an RNA molecule comprising (i) a complementary region that hybridizes with a target mRNA and (ii) a poly(A) region.

In some embodiments, provided herein are expression vectors (e.g., a single vector or a plurality of vectors) including any one(s) of the recombinant expression systems described herein. In some embodiments, a provided expression vector is a viral vector. In some embodiments, such a viral vector is an adeno-associated viral vector (AAV), a lentiviral vector, or an adenoviral vector.

In some embodiments, provided herein are cells comprising one or more any one of the provided nucleic acid agents and/or recombinant expression systems (e.g., one or more of the expression vectors described herein).

In some embodiments, a provided nucleic acid agent can include about 50 to about 200 (e.g., about 50 to about 180, about 50 to about 160, about 50 to about 150, about 50 to about 140, about 50 to about 120, about 50 to about 100, about 50 to about 90, about 50 to about 80, about 50 to about 70, about 50 to about 60, about 60 to about 200, about 60 to about 180, about 60 to about 160, about 60 to about 150, about 60 to about 140, about 60 to about 120, about 60 to about 100, about 60 to about 90, about 60 to about 80, about 60 to about 70, about 70 to about 200, about 70 to about 180, about 70 to about 160, about 70 to about 150, about 70 to about 140, about 70 to about 120, about 70 to about 100, about 70 to about 90, about 70 to about 80, about 80 to about 200, about 80 to about 180, about 80 to about 160, about 80 to about 150, about 80 to about 140, about 80 to about 120, about 80 to about 100, about 80 to about 90, about 90 to about 200, about 90 to about 180, about 90 to about 160, about 90 to about 150, about 90 to about 140, about 90 to about 120, about 90 to about 100, about 100 to about 200, about 100 to about 180, about 100 to about 160, about 100 to about 150, about 100 to about 140, about 100 to about 120, about 120 to about 200, about 120 to about 180, about 120 to about 160, about 120 to about 150, about 120 to about 140, about 140 to about 200, about 140 to about 180, about 140 to about 160, about 140 to about 150, about 150 to about 200, about 150 to about 180, about 150 to about 160, about 160 to about 200, about 160 to about 180, or about 180 to about 200) nucleic acid residues (e.g., nucleotides), e.g., as a single nucleic acid molecule, or as two or more distinct nucleic acid molecules associated with one another. In some embodiments, a provided nucleic acid agent can include about 100 to about 150 nucleic acid residues (e.g., nucleotides).

In some embodiments, a provided nucleic acid agent comprises or consists of natural nucleic acid residues. In some embodiments, a provided nucleic acid agent comprises or consists of one or more nucleic acid residue analogs (e.g., one or more nucleotide analogs—for example one or more sugar and/or base analogs and/or one or more internucleoside linkages that is not a phosphodiester bond.

In some embodiments, a provided nucleic acid agent comprises a nucleotide modification. In some embodiments, a nucleotide modification is a N1-methyladenosine (m1A), N6-methyladenosine (m6A), or adenosine to inosine (A-to-I) modification. In some embodiments, a nucleotide modification is a pseudouracil, M1-pseudouracil, 5-methoxyuridine (5moU), or N4-acetylcytidine. In some embodiments, a nucleotide modification is a 1,2′-O-dimethyladenosine (m1Am), 1,2′-O-dimethylguanosine (m1Gm), 1,2′-O-dimethylinosine (m1Im), 1-methyl-3-(3-amino-3-carboxypropyl)pseudouridine (m1acp3Y), 1-methyladenosine (m1A), 1-methylguanosine (m1G), 1-methylinosine (m1I), 1-methylpseudouridine (m1Y), 2,8-dimethyladenosine (m2,8A), 2-geranylthiouridine (ges2U), 2-lysidine (k2C), 2-methyladenosine (m2A), 2-methylthiomethylenethio-N6-isopentenyl-adenosine (msms2i6A), 2-methylthio-cyclic-N6-threonylcarbamoyladenosine (ms2ct6A), 2-methylthio-N6-(cis-hydroxyisopentenyl)-adenosine (ms2io6A), 2-methylthio-N6-hydroxynorvalylcarbamoyladenosine (ms2hn6A), 2-methylthio-N6-isopentenyladenosine (ms2i6A), 2-methylthio-N6-methyladenosine (ms2m6A), 2-methylthio-N6-threonylcarbamoyladenosine (ms2t6A), 2-selenouridine (se2U), 2-thio-2′-O-methyluridine (s2Um), 2-thiocytidine (s2C), 2-thiouridine (s2U), 2′-O-methyladenosine (Am), 2′-O-methylcytidine (Cm), 2′-O-methylguanosine (Gm), 2′-O-methylinosine (Im), 2′-O-methylpseudouridine (Ym), 2′-O-methyluridine (Um), 2′-O-methyluridine 5-oxyacetic acid methyl ester (mcmo5Um), 2′-O-ribosyladenosine (phosphate) (Ar(p)), 2′-O-ribosylguanosine (phosphate) (Gr(p)), 3,2′-O-dimethyluridine (m3Um), 3-(3-amino-3-carboxypropyl)-5,6-dihydrouridine (acp3D), 3-(3-amino-3-carboxypropyl)pseudouridine (acp3Y), 3-(3-amino-3-carboxypropyl)uridine (acp3U), 3-methylcytidine (m3C), 3-methylpseudouridine (m3Y), 3-methyluridine (m3U), 4-demethylwyosine (imG-14), 4-thiouridine (s4U), 5,2′-O-dimethylcytidine (m5Cm), 5,2′-O-dimethyluridine (m5Um), 5-(carboxyhydroxymethyl)-2′-O-methyluridine methyl ester (mchm5Um), 5-(carboxyhydroxymethyl)uridine methyl ester (mchm5U), 5-(isopentenylaminomethyl)-2-thiouridine (inm5s2U), 5-(isopentenylaminomethyl)-2′-O-methyluridine (inm5Um), 5-(isopentenylaminomethyl)uridine (inm5U), 5-aminomethyl-2-geranylthiouridine (nm5ges2U), 5-aminomethyl-2-selenouridine (nm5se2U), 5-aminomethyl-2-thiouridine (nm5s2U), 5-aminomethyluridine (nm5U), 5-carbamoylhydroxymethyluridine (nchm5U), 5-carbamoylmethyl-2-thiouridine (ncm5s2U), 5-carbamoylmethyl-2′-O-methyluridine (ncm5Um), 5-carbamoylmethyluridine (ncm5U), 5-carboxyhydroxymethyluridine (chm5U), 5-carboxymethyl-2-thiouridine (cm5s2U), 5-carboxymethylaminomethyl-2-geranylthiouridine (cmnm5ges2U), 5-carboxymethylaminomethyl-2-selenouridine (cmnm5se2U), 5-carboxymethylaminomethyl-2-thiouridine (cmnm5s2U), 5-carboxymethylaminomethyl-2′-O-methyluridine (cmnm5Um), 5-carboxymethylaminomethyluridine (cmnm5U), 5-carboxymethyluridine (cm5U), 5-cyanomethyluridine (cnm5U), 5-formyl-2′-O-methylcytidine (f5Cm), 5-formylcytidine (f5C), 5-hydroxycytidine (ho5C), 5-hydroxymethylcytidine (hm5C), 5-hydroxyuridine (ho5U), 5-methoxycarbonylmethyl-2-thiouridine (mcm5s2U), 5-methoxycarbonylmethyl-2′-O-methyluridine (mcm5Um), 5-methoxycarbonylmethyluridine (mcm5U), 5-methoxyuridine (mo5U), 5-methyl-2-thiouridine (m5s2U), 5-methylaminomethyl-2-geranylthiouridine (mnm5ges2U), 5-methylaminomethyl-2-selenouridine (mnm5se2U), 5-methylaminomethyl-2-thiouridine (mnm5s2U), 5-methylaminomethyluridine (mnm5U), 5-methylcytidine (m5C), 5-methyldihydrouridine (m5D), 5-methyluridine (m5U), 5-taurinomethyl-2-thiouridine (tm5s2U), 5-taurinomethyluridine (tm5U), 7-aminocarboxypropyl-demethylwyosine (yW-86), 7-aminocarboxypropylwyosine (yW-72), 7-aminocarboxypropylwyosine methyl ester (yW-58), 7-aminomethyl-7-deazaguanosine (preQ1tRNA), 7-cyano-7-deazaguanosine (preQOtRNA), 7-methylguanosine (m7G), 8-methyladenosine (m8A), N2,2′-O-dimethylguanosine (m2Gm), N2,7,2′-O-trimethylguanosine (m2,7Gm), N2,7-dimethylguanosine (m2,7G), N2,N2,2′-O-trimethylguanosine (m2,2Gm), N2,N2,7-trimethylguanosine (m2,2,7G), N2,N2-dimethylguanosine (m2,2G), N2-methylguanosine (m2G), N4,2′-O-dimethylcytidine (m4Cm), N4,N4,2′-O-trimethylcytidine (m4,4Cm), N4,N4-dimethylcytidine (m4,4C), N4-acetyl-2′-O-methylcytidine (ac4Cm), N4-acetylcytidine (ac4C), N4-methylcytidine (m4C), N6,2′-O-dimethyladenosine (m6Am), N6,N6,2′-O-trimethyladenosine (m6,6Am), N6,N6-dimethyladenosine (m6,6A), N6-(cis-hydroxyisopentenyl)adenosine (io6A), N6-acetyladenosine (ac6A), N6-formyladenosine (f6A), N6-glycinylcarbamoyladenosine (g6A), N6-hydroxymethyladenosine (hm6A), N6-hydroxynorvalylcarbamoyladenosine (hn6A), N6-isopentenyladenosine (i6A), N6-methyl-N6-threonylcarbamoyladenosine (m6t6A), N6-methyladenosine (m6A), N6-threonylcarbamoyladenosine (t6A), Agmatidine (C+), Archaeosine (G+), cyclic N6-threonylcarbamoyladenosine (ct6A), dihydrouridine (D), epoxyqueuosine (oQtRNA), galactosyl-queuosine (galQtRNA), glutamyl-queuosine (gluQtRNA), hydroxy-N6-threonylcarbamoyladenosine (ht6A), hydroxywybutosine (OHyW), inosine (I), isowyosine (imG2), mannosyl-queuosine (manQtRNA), methylated undermodified hydroxywybutosine (OHyWy), methylwyosine (mimG), peroxywybutosine (o2yW), pseudouridine (Y), queuosine (QtRNA), undermodified hydroxywybutosine (OHyWx), uridine 5-oxyacetic acid (cmo5U), uridine 5-oxyacetic acid methyl ester (mcmo5U), wybutosine (yW), or wyosine (imG) modification.

Alternatively or additionally, in some embodiments, a provided nucleic acid agent (and/or an element thereof) is or comprises a single stranded nucleic acid. In some embodiments, a provided nucleic acid agent (and/or an element thereof) is or comprises ribonucleic acid (RNA), deoxyribonucleic acid (DNA), threose nucleic acid (TNA), glycol nucleic acid (GNA), peptide nucleic acid (PNA), locked nucleic acid (LNA, such as for example, LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or a combination (e.g., “hybrid”) thereof. In some embodiments, a provided nucleic acid agent is a single RNA strand. In some embodiments, a provided nucleic acid agent is a single DNA strand. In some embodiments, a provided nucleic acid agent is a single strand that includes one or more, or all, modified nucleotides.

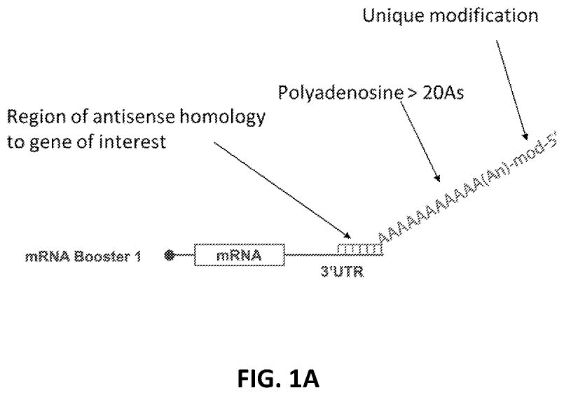

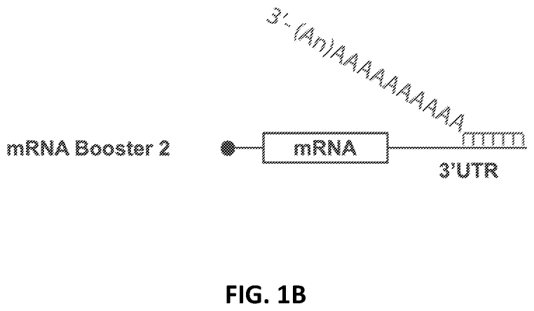

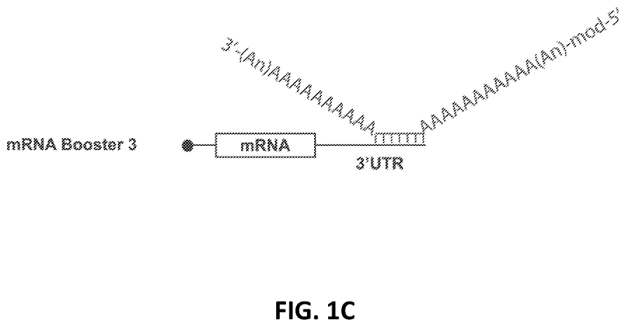

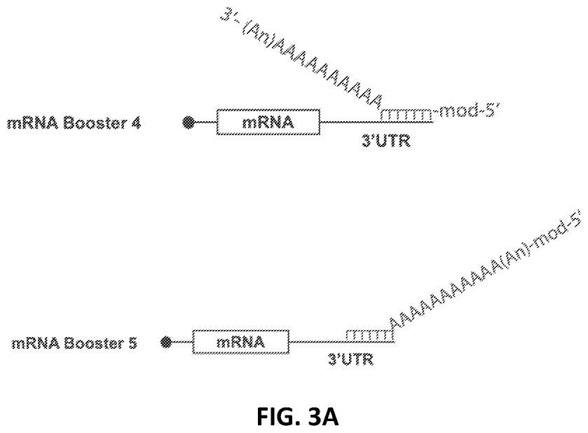

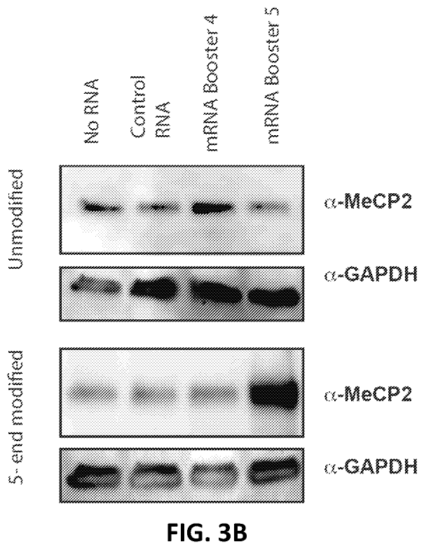

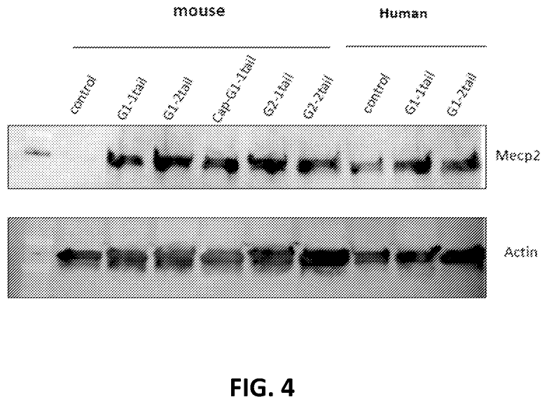

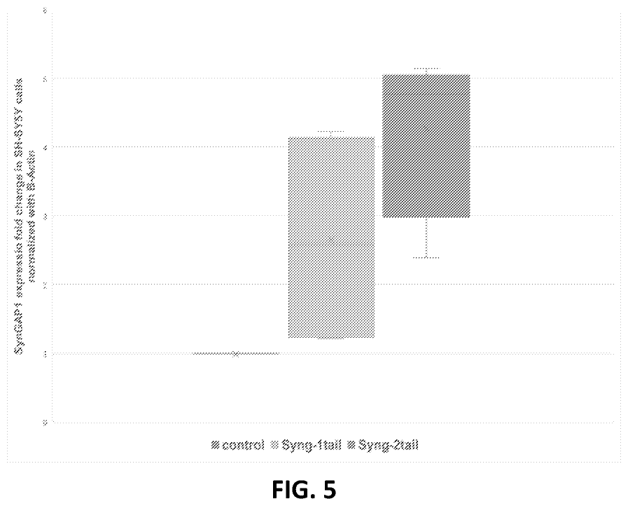

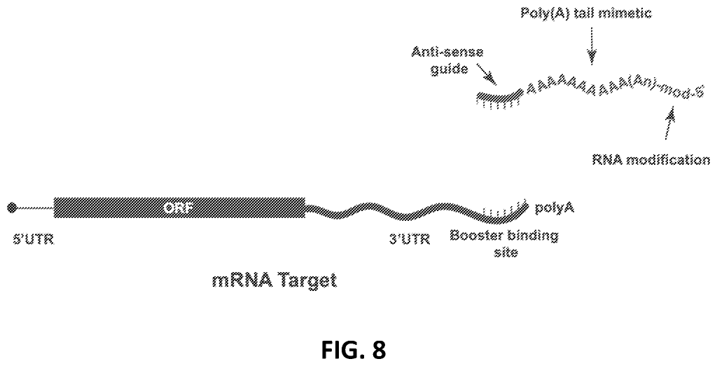

In some embodiments, a nucleic acid agent can include a complementary element that hybridizes with a target mRNA and a poly(A) element. (see, for example, exemplary agents as depicted in one or more of FIG. 1A, FIG. 1B, FIG. 1C, FIG. 3A, and/or FIG. 8).

### Complementary Element

A provided nucleic acid agent, as described herein, typically includes a “complementary element” (e.g., “complementary region”) designed and/or constructed based on nucleobase complementarity. In some embodiments, a complementary element includes a nucleic acid sequence that is complementary to an RNA (e.g., target mRNA). In some embodiments, a complementary element can hybridize to a target mRNA. In some embodiments, a complementary element hybridizes to a 3′ untranslated region of the target mRNA. In some embodiments, a complementary element can include about 30 (e.g., about 15, about 20, about 25, about 35, about 40, about 45, or about 50) nucleotides. In some embodiments, a complementary element comprises RNA. In some embodiments, a complementary element comprises DNA. In some embodiments, the complementary element comprises both RNA and DNA.

In some embodiments, a complementary element comprises a nucleotide modification. In some embodiments, a nucleotide modification is a N1-methyladenosine (m1A), N6-methyladenosine (m6A), or adenosine to inosine (A-to-I) modification. In some embodiments, a nucleotide modification is a pseudouracil, M1-pseudouracil, 5-methoxyuridine (5moU), or N4-acetylcytidine. In some embodiments, a nucleotide modification is a 1,2′-O-dimethyladenosine (m1Am), 1,2′-O-dimethylguanosine (m1Gm), 1,2′-O-dimethylinosine (m1Im), 1-methyl-3-(3-amino-3-carboxypropyl)pseudouridine (m1acp3Y), 1-methyladenosine (m1A), 1-methylguanosine (m1G), 1-methylinosine (m1I), 1-methylpseudouridine (m1Y), 2,8-dimethyladenosine (m2,8A), 2-geranylthiouridine (ges2U), 2-lysidine (k2C), 2-methyladenosine (m2A), 2-methylthiomethylenethio-N6-isopentenyl-adenosine (msms2i6A), 2-methylthio-cyclic-N6-threonylcarbamoyladenosine (ms2ct6A), 2-methylthio-N6-(cis-hydroxyisopentenyl)-adenosine (ms2io6A), 2-methylthio-N6-hydroxynorvalylcarbamoyladenosine (ms2hn6A), 2-methylthio-N6-isopentenyladenosine (ms2i6A), 2-methylthio-N6-methyladenosine (ms2m6A), 2-methylthio-N6-threonylcarbamoyladenosine (ms2t6A), 2-selenouridine (se2U), 2-thio-2′-O-methyluridine (s2Um), 2-thiocytidine (s2C), 2-thiouridine (s2U), 2′-O-methyladenosine (Am), 2′-O-methylcytidine (Cm), 2′-O-methylguanosine (Gm), 2′-O-methylinosine (Im), 2′-O-methylpseudouridine (Ym), 2′-O-methyluridine (Um), 2′-O-methyluridine 5-oxyacetic acid methyl ester (mcmo5Um), 2′-O-ribosyladenosine (phosphate) (Ar(p)), 2′-O-ribosylguanosine (phosphate) (Gr(p)), 3,2′-O-dimethyluridine (m3Um), 3-(3-amino-3-carboxypropyl)-5,6-dihydrouridine (acp3D), 3-(3-amino-3-carboxypropyl)pseudouridine (acp3Y), 3-(3-amino-3-carboxypropyl)uridine (acp3U), 3-methylcytidine (m3C), 3-methylpseudouridine (m3Y), 3-methyluridine (m3U), 4-demethylwyosine (imG-14), 4-thiouridine (s4U), 5,2′-O-dimethylcytidine (m5Cm), 5,2′-O-dimethyluridine (m5Um), 5-(carboxyhydroxymethyl)-2′-O-methyluridine methyl ester (mchm5Um), 5-(carboxyhydroxymethyl)uridine methyl ester (mchm5U), 5-(isopentenylaminomethyl)-2-thiouridine (inm5s2U), 5-(isopentenylaminomethyl)-2′-O-methyluridine (inm5Um), 5-(isopentenylaminomethyl)uridine (inm5U), 5-aminomethyl-2-geranylthiouridine (nm5ges2U), 5-aminomethyl-2-selenouridine (nm5se2U), 5-aminomethyl-2-thiouridine (nm5s2U), 5-aminomethyluridine (nm5U), 5-carbamoylhydroxymethyluridine (nchm5U), 5-carbamoylmethyl-2-thiouridine (ncm5s2U), 5-carbamoylmethyl-2′-O-methyluridine (ncm5Um), 5-carbamoylmethyluridine (ncm5U), 5-carboxyhydroxymethyluridine (chm5U), 5-carboxymethyl-2-thiouridine (cm5s2U), 5-carboxymethylaminomethyl-2-geranylthiouridine (cmnm5ges2U), 5-carboxymethylaminomethyl-2-selenouridine (cmnm5se2U), 5-carboxymethylaminomethyl-2-thiouridine (cmnm5s2U), 5-carboxymethylaminomethyl-2′-O-methyluridine (cmnm5Um), 5-carboxymethylaminomethyluridine (cmnm5U), 5-carboxymethyluridine (cm5U), 5-cyanomethyluridine (cnm5U), 5-formyl-2′-O-methylcytidine (f5Cm), 5-formylcytidine (f5C), 5-hydroxycytidine (ho5C), 5-hydroxymethylcytidine (hm5C), 5-hydroxyuridine (ho5U), 5-methoxycarbonylmethyl-2-thiouridine (mcm5s2U), 5-methoxycarbonylmethyl-2′-O-methyluridine (mcm5Um), 5-methoxycarbonylmethyluridine (mcm5U), 5-methoxyuridine (mo5U), 5-methyl-2-thiouridine (m5s2U), 5-methylaminomethyl-2-geranylthiouridine (mnm5ges2U), 5-methylaminomethyl-2-selenouridine (mnm5se2U), 5-methylaminomethyl-2-thiouridine (mnm5s2U), 5-methylaminomethyluridine (mnm5U), 5-methylcytidine (m5C), 5-methyldihydrouridine (m5D), 5-methyluridine (m5U), 5-taurinomethyl-2-thiouridine (tm5s2U), 5-taurinomethyluridine (tm5U), 7-aminocarboxypropyl-demethylwyosine (yW-86), 7-aminocarboxypropylwyosine (yW-72), 7-aminocarboxypropylwyosine methyl ester (yW-58), 7-aminomethyl-7-deazaguanosine (preQ1tRNA), 7-cyano-7-deazaguanosine (preQOtRNA), 7-methylguanosine (m7G), 8-methyladenosine (m8A), N2,2′-O-dimethylguanosine (m2Gm), N2,7,2′-O-trimethylguanosine (m2,7Gm), N2,7-dimethylguanosine (m2,7G), N2,N2,2′-O-trimethylguanosine (m2,2Gm), N2,N2,7-trimethylguanosine (m2,2,7G), N2,N2-dimethylguanosine (m2,2G), N2-methylguanosine (m2G), N4,2′-O-dimethylcytidine (m4Cm), N4,N4,2′-O-trimethylcytidine (m4,4Cm), N4,N4-dimethylcytidine (m4,4C), N4-acetyl-2′-O-methylcytidine (ac4Cm), N4-acetylcytidine (ac4C), N4-methylcytidine (m4C), N6,2′-O-dimethyladenosine (m6Am), N6,N6,2′-O-trimethyladenosine (m6,6Am), N6,N6-dimethyladenosine (m6,6A), N6-(cis-hydroxyisopentenyl)adenosine (io6A), N6-acetyladenosine (ac6A), N6-formyladenosine (f6A), N6-glycinylcarbamoyladenosine (g6A), N6-hydroxymethyladenosine (hm6A), N6-hydroxynorvalylcarbamoyladenosine (hn6A), N6-isopentenyladenosine (i6A), N6-methyl-N6-threonylcarbamoyladenosine (m6t6A), N6-methyladenosine (m6A), N6-threonylcarbamoyladenosine (t6A), Agmatidine (C+), Archaeosine (G+), cyclic N6-threonylcarbamoyladenosine (ct6A), dihydrouridine (D), epoxyqueuosine (oQtRNA), galactosyl-queuosine (galQtRNA), glutamyl-queuosine (gluQtRNA), hydroxy-N6-threonylcarbamoyladenosine (ht6A), hydroxywybutosine (OHyW), inosine (I), isowyosine (imG2), mannosyl-queuosine (manQtRNA), methylated undermodified hydroxywybutosine (OHyWy), methylwyosine (mimG), peroxywybutosine (o2yW), pseudouridine (Y), queuosine (QtRNA), undermodified hydroxywybutosine (OHyWx), uridine 5-oxyacetic acid (cmo5U), uridine 5-oxyacetic acid methyl ester (mcmo5U), wybutosine (yW), or wyosine (imG) modification.

In some embodiments, a target mRNA is an mRNA that encodes a polypeptide of interest (e.g., a polypeptide whose increased expression provides an activity or benefit of interest, for example to or in a cell, tissue, system or organism in which it is expressed.

In some embodiments, a target mRNA is an mRNA of an active allele of a gene associated with a disorder associated with a decrease in the expression of a protein from the mRNA. In some embodiments, such a disorder is a haploinsufficiency disorder. In some embodiments, a target mRNA encodes the MeCP2 protein. In some embodiments, a nucleic acid agent is or comprises a nucleic acid molecule that comprises or consists of SEQ ID NO: 1.

Complementary region targeting 3′ UTR of MeCP2
SEQ ID NO: 1
CCACAGGCTAAAAATGTATATGCCCAAAGA

In some embodiments, a target mRNA encodes a reduced-activity variant of a polypeptide of interest. In some embodiments, a target mRNA may be exogenous to a cell, tissue, organism or other system of interest. For example, in some embodiments, a target mRNA may be a therapeutic mRNA and/or may be a transcript of a gene included in or otherwise provided by a gene therapy or cell therapy agent. In some particular embodiments, a target mRNA may encode a toxic or suicide polypeptide, for example whose expression reduces viability of (e.g., by inducing or promoting cell death apoptosis or cell death) a cell in which it is expressed or with which it otherwise comes into contact. In some particular such embodiments, such a cell may be a diseased cell (e.g., a cancer cell); alternatively or additionally, in some particular such embodiments, such a cell may be a cell therapy product (e.g., a preparation of engineered T cells such as a CAR-T cells, or of engineered NK cells, or of engineered B cells, etc).

### Poly(A) Element

A “poly(A) region” included in a nucleic acid agent as described and/or utilized herein typically is or comprises a chain of adenosine residues.

In some embodiments, a nucleic acid agent is arranged and constructed so that a poly(A) element is located 5′ to a complementary element. For example, in some embodiments, a nucleic acid agent is or comprises a nucleic acid molecule that includes a poly(A) region located 5′ to a complementary region.

In some embodiments, a nucleic acid agent is arranged and constructed so that a poly(A) element is located 3′ to a complementary element. For example, in some embodiments, a nucleic acid agent is or comprises a nucleic acid molecule that includes a poly(A) region located 3′ to a complementary region.

In some embodiments, a nucleic acid agent is arranged and constructed so that a poly(A) element is located 5′ to a complementary element, and the nucleic acid agent further includes a cap element located 5′ to the complementary element. For example, in some embodiments, a nucleic acid agent is or comprises a nucleic acid molecule that includes a poly(A) region is located 3′ to the complementary region and that further comprises a 5′ cap region located 5′ to the complementary region.

In some embodiments, a nucleic acid agent is arranged and constructed so that a first poly(A) element is located 5′ to a complementary element and a second poly(A) region is located 5′ to a complementary region. For example, in some embodiments, a nucleic acid agent is or comprises a nucleic acid molecule that comprises a first poly(A) region located 5′ to the complementary region and a second poly(A) region located 3′ to the complementary region.

Those skilled in the art will be familiar with the term “poly(A) tail”, which typically refers to stretch of adenosine residues located at the end of an mRNA. In wild-type situations, a poly(A) tail is located at the 3′ end of a mRNA, and is post-transcriptionally synthesized on mRNAs that include a polyadenylation (poly(A)) signal sequence. The term “poly(A) signal sequence” or “poly(A) signal” is commonly used to refer to a sequence that triggers the endonuclease cleavage of an mRNA, and the addition of a sequence of adenosines to the 3′end of the cleaved mRNA. Non-limiting examples of poly(A) signals include: bovine growth hormone (bGH) poly(A) signal, human growth hormone (hGH) poly(A) signal. Additional examples of poly(A) signal sequences are known in the art.

Poly(A) tails or poly(A) elements (e.g., poly(A) regions) can function by binding poly(A) binding protein (PABP). PABP is a highly conserved RNA binding protein in eukaryotes. This protein has four N-terminal RNA recognition motif (RRM) domains, which bind poly(A) RNA with a nanomolar affinity. The RRMs are followed by a proline-rich linker and a C-terminal MLLE domain. The MLLE domain recognizes a peptide motif called poly(A)-interacting motif 2 (PAM2), which is found in a number of PABP partner proteins that regulate mRNA metabolism (stability and translation). The presence of PABP on mRNA is known to stimulate their activity, enhancing translation and mRNA stability. In some embodiments, a poly(A) element in a provide nucleic acid agent (e.g., a poly(A) region of a provided nucleic acid molecule) can function by recruiting translation stimulatory factors (e.g., PABC1). In some embodiments, a poly(A) element (e.g., a poly(A) region) can be resistant to deadenylation; without wishing to be bound by any particular theory, we propose a poly(A) element (e.g., a poly(A) region) may thus maintain a target mRNA in a positive translational state.

Poly(A) tails or poly(A) elements (e.g., poly(A) regions) can be added to most nascent eukaryotic messenger RNAs (mRNAs) at their 3′ end during a complex process that includes cleavage of the primary transcript and a coupled polyadenylation reaction driven by the poly(A) signal sequence. The term “polyadenylation” refers to the covalent linkage of a polyadenylyl moiety, or a modified variant thereof, to the 3′ end of an mRNA molecule.

In some embodiments, a gene delivery vector provided and/or utilized in accordance with the present disclosure can include a sequence encoding a poly(A) region proximal to a sequence encoding a complementary region. In some embodiments, a gene delivery vector can include a poly(T) sequence proximal to a sequence encoding a complementary region, wherein the poly(T) sequence encodes a poly(A) region. In some embodiments, a gene delivery vector can include a sequence comprising a poly(A) region at the end of an isolated nucleic acid encoding a complementary region.

In some embodiments, the poly(A) element (e.g., a poly(A) region) includes about 20 to about 80 (e.g., about 25 to about 80, about 30 to about 80, about 35 to about 80, about 40 to about 80, about 45 to about 80, about 50 to about 80, about 55 to about 80, about 60 to about 80, about 65 to about 80, about 70 to about 80, about 75 to about 80, about 20 to about 75, about 25 to about 75, about 30 to about 75, about 35 to about 75, about 40 to about 75, about 45 to about 75, about 50 to about 75, about 55 to about 75, about 60 to about 75, about 65 to about 75, about 70 to about 75, about 20 to about 70, about 25 to about 70, about 30 to about 70, about 35 to about 70, about 40 to about 70, about 45 to about 70, about 50 to about 70, about 55 to about 70, about 60 to about 70, about 65 to about 70, about 20 to about 65, about 25 to about 65, about 30 to about 65, about 35 to about 65, about 40 to about 65, about 45 to about 65, about 50 to about 65, about 55 to about 65, about 60 to about 65, about 20 to about 60, about 25 to about 60, about 30 to about 60, about 35 to about 60, about 40 to about 60, about 45 to about 60, about 50 to about 60, about 55 to about 60, about 20 to about 55, about 25 to about 55, about 30 to about 55, about 35 to about 55, about 40 to about 55, about 45 to about 55, about 50 to about 55, about 20 to about 50, about 25 to about 50, about 30 to about 50, about 35 to about 50, about 40 to about 50, about 45 to about 50, about 20 to about 45, about 25 to about 45, about 30 to about 45, about 35 to about 45, about 40 to about 45, about 20 to about 40, about 25 to about 40, about 30 to about 40, about 35 to about 40, about 20 to about 35, about 25 to about 35, about 30 to about 35, about 20 to about 30, about 25 to about 30, or about 20 to about 25) adenine nucleotides. In some embodiments, a poly(A) element (e.g., a poly(A) region) comprises about 20 nucleotides. In some embodiments, a poly(A) element (e.g., a poly(A) region) comprises about 50 nucleotides. In some embodiments, a poly(A) element (e.g., a poly(A) region) comprises about 75 nucleotides.

In some embodiments, a poly(A) element (e.g., a poly(A) region) can include a non-adenine nucleotide.

In some embodiments, a poly(A) element in a nucleic acid agent (e.g., a poly(A) region in a nucleic acid molecule) provided and/or utilized in accordance with the present disclosure comprises a nucleotide modification. In some embodiments, the poly(A) region comprises a nucleotide modification. In some embodiments, a nucleotide modification is a N1-methyladenosine (m1A), N6-methyladenosine (m6A), or adenosine to inosine (A-to-I) modification. In some embodiments, a nucleotide modification is a pseudouracil, M1-pseudouracil, 5-methoxyuridine (5moU), or N4-acetylcytidine. In some embodiments, the nucleotide modification is a 1,2′-O-dimethyladenosine (m1Am), 1,2′-O-dimethylguanosine (m1Gm), 1,2′-O-dimethylinosine (m1Im), 1-methyl-3-(3-amino-3-carboxypropyl)pseudouridine (m1acp3Y), 1-methyladenosine (m1A), 1-methylguanosine (m1G), 1-methylinosine (m1I), 1-methylpseudouridine (m1Y), 2,8-dimethyladenosine (m2,8A), 2-geranylthiouridine (ges2U), 2-lysidine (k2C), 2-methyladenosine (m2A), 2-methylthiomethylenethio-N6-isopentenyl-adenosine (msms2i6A), 2-methylthio-cyclic-N6-threonylcarbamoyladenosine (ms2ct6A), 2-methylthio-N6-(cis-hydroxyisopentenyl)-adenosine (ms2io6A), 2-methylthio-N6-hydroxynorvalylcarbamoyladenosine (ms2hn6A), 2-methylthio-N6-isopentenyladenosine (ms2i6A), 2-methylthio-N6-methyladenosine (ms2m6A), 2-methylthio-N6-threonylcarbamoyladenosine (ms2t6A), 2-selenouridine (se2U), 2-thio-2′-O-methyluridine (s2Um), 2-thiocytidine (s2C), 2-thiouridine (s2U), 2′-O-methyladenosine (Am), 2′-O-methylcytidine (Cm), 2′-O-methylguanosine (Gm), 2′-O-methylinosine (Im), 2′-O-methylpseudouridine (Ym), 2′-O-methyluridine (Um), 2′-O-methyluridine 5-oxyacetic acid methyl ester (mcmo5Um), 2′-O-ribosyladenosine (phosphate) (Ar(p)), 2′-O-ribosylguanosine (phosphate) (Gr(p)), 3,2′-O-dimethyluridine (m3Um), 3-(3-amino-3-carboxypropyl)-5,6-dihydrouridine (acp3D), 3-(3-amino-3-carboxypropyl)pseudouridine (acp3Y), 3-(3-amino-3-carboxypropyl)uridine (acp3U), 3-methylcytidine (m3C), 3-methylpseudouridine (m3Y), 3-methyluridine (m3U), 4-demethylwyosine (imG-14), 4-thiouridine (s4U), 5,2′-O-dimethylcytidine (m5Cm), 5,2′-O-dimethyluridine (m5Um), 5-(carboxyhydroxymethyl)-2′-O-methyluridine methyl ester (mchm5Um), 5-(carboxyhydroxymethyl)uridine methyl ester (mchm5U), 5-(isopentenylaminomethyl)-2-thiouridine (inm5s2U), 5-(isopentenylaminomethyl)-2′-O-methyluridine (inm5Um), 5-(isopentenylaminomethyl)uridine (inm5U), 5-aminomethyl-2-geranylthiouridine (nm5ges2U), 5-aminomethyl-2-selenouridine (nm5se2U), 5-aminomethyl-2-thiouridine (nm5s2U), 5-aminomethyluridine (nm5U), 5-carbamoylhydroxymethyluridine (nchm5U), 5-carbamoylmethyl-2-thiouridine (nem5s2U), 5-carbamoylmethyl-2′-O-methyluridine (ncm5Um), 5-carbamoylmethyluridine (ncm5U), 5-carboxyhydroxymethyluridine (chm5U), 5-carboxymethyl-2-thiouridine (cm5s2U), 5-carboxymethylaminomethyl-2-geranylthiouridine (cmnm5ges2U), 5-carboxymethylaminomethyl-2-selenouridine (cmnm5se2U), 5-carboxymethylaminomethyl-2-thiouridine (cmnm5s2U), 5-carboxymethylaminomethyl-2′-O-methyluridine (cmnm5Um), 5-carboxymethylaminomethyluridine (cmnm5U), 5-carboxymethyluridine (cm5U), 5-cyanomethyluridine (cnm5U), 5-formyl-2′-O-methylcytidine (f5Cm), 5-formylcytidine (f5C), 5-hydroxycytidine (ho5C), 5-hydroxymethylcytidine (hm5C), 5-hydroxyuridine (ho5U), 5-methoxycarbonylmethyl-2-thiouridine (mcm5s2U), 5-methoxycarbonylmethyl-2′-O-methyluridine (mcm5Um), 5-methoxycarbonylmethyluridine (mcm5U), 5-methoxyuridine (mo5U), 5-methyl-2-thiouridine (m5s2U), 5-methylaminomethyl-2-geranylthiouridine (mnm5ges2U), 5-methylaminomethyl-2-selenouridine (mnm5se2U), 5-methylaminomethyl-2-thiouridine (mnm5s2U), 5-methylaminomethyluridine (mnm5U), 5-methylcytidine (m5C), 5-methyldihydrouridine (m5D), 5-methyluridine (m5U), 5-taurinomethyl-2-thiouridine (tm5s2U), 5-taurinomethyluridine (tm5U), 7-aminocarboxypropyl-demethylwyosine (yW-86), 7-aminocarboxypropylwyosine (yW-72), 7-aminocarboxypropylwyosine methyl ester (yW-58), 7-aminomethyl-7-deazaguanosine (preQ1tRNA), 7-cyano-7-deazaguanosine (preQOtRNA), 7-methylguanosine (m7G), 8-methyladenosine (m8A), N2,2′-O-dimethylguanosine (m2Gm), N2,7,2′-O-trimethylguanosine (m2,7Gm), N2,7-dimethylguanosine (m2,7G), N2,N2,2′-O-trimethylguanosine (m2,2Gm), N2,N2,7-trimethylguanosine (m2,2,7G), N2,N2-dimethylguanosine (m2,2G), N2-methylguanosine (m2G), N4,2′-O-dimethylcytidine (m4Cm), N4,N4,2′-O-trimethylcytidine (m4,4Cm), N4,N4-dimethylcytidine (m4,4C), N4-acetyl-2′-O-methylcytidine (ac4Cm), N4-acetylcytidine (ac4C), N4-methylcytidine (m4C), N6,2′-O-dimethyladenosine (m6Am), N6,N6,2′-O-trimethyladenosine (m6,6Am), N6,N6-dimethyladenosine (m6,6A), N6-(cis-hydroxyisopentenyl)adenosine (io6A), N6-acetyladenosine (ac6A), N6-formyladenosine (f6A), N6-glycinylcarbamoyladenosine (g6A), N6-hydroxymethyladenosine (hm6A), N6-hydroxynorvalylcarbamoyladenosine (hn6A), N6-isopentenyladenosine (i6A), N6-methyl-N6-threonylcarbamoyladenosine (m6t6A), N6-methyladenosine (m6A), N6-threonylcarbamoyladenosine (t6A), Agmatidine (C+), Archaeosine (G+), cyclic N6-threonylcarbamoyladenosine (ct6A), dihydrouridine (D), epoxyqueuosine (oQtRNA), galactosyl-queuosine (galQtRNA), glutamyl-queuosine (gluQtRNA), hydroxy-N6-threonylcarbamoyladenosine (ht6A), hydroxywybutosine (OHyW), inosine (I), isowyosine (imG2), mannosyl-queuosine (manQtRNA), methylated undermodified hydroxywybutosine (OHyWy), methylwyosine (mimG), peroxywybutosine (o2yW), pseudouridine (Y), queuosine (QtRNA), undermodified hydroxywybutosine (OHyWx), uridine 5-oxyacetic acid (cmo5U), uridine 5-oxyacetic acid methyl ester (mcmo5U), wybutosine (yW), or wyosine (imG) modification.

### 5′ Cap Element

In some embodiments, a nucleic acid agent (e.g., that is or comprises a nucleic acid molecule) further comprises a 5′ cap element.

As used herein, the term “5′ cap element” typically refers to a modified nucleotide on the 5′ end of a nucleic acid molecule. In some embodiments, a 5′ cap element can include a nucleotide modification. In some embodiments, the nucleotide modification is a N1-methyladenosine (m1A), N6-methyladenosine (m6A), or adenosine to inosine (A-to-I) modification. In some embodiments, the nucleotide modification is a pseudouracil, M1-pseudouracil, 5-methoxyuridine (5moU), or N4-acetylcytidine. In some embodiments, a 5′ cap region comprises a eukaryotic 5′ cap structure. In some embodiments, a 5′ cap region can include a G(5′)ppp(5′)G sequence. In some embodiments, the nucleotide modification is a 1,2′-O-dimethyladenosine (m1Am), 1,2′-O-dimethylguanosine (m1Gm), 1,2′-O-dimethylinosine (m1Im), 1-methyl-3-(3-amino-3-carboxypropyl)pseudouridine (m1acp3Y), 1-methyladenosine (m1A), 1-methylguanosine (m1G), 1-methylinosine (m1I), 1-methylpseudouridine (m1Y), 2,8-dimethyladenosine (m2,8A), 2-geranylthiouridine (ges2U), 2-lysidine (k2C), 2-methyladenosine (m2A), 2-methylthiomethylenethio-N6-isopentenyl-adenosine (msms2i6A), 2-methylthio-cyclic-N6-threonylcarbamoyladenosine (ms2ct6A), 2-methylthio-N6-(cis-hydroxyisopentenyl)-adenosine (ms2io6A), 2-methylthio-N6-hydroxynorvalylcarbamoyladenosine (ms2hn6A), 2-methylthio-N6-isopentenyladenosine (ms2i6A), 2-methylthio-N6-methyladenosine (ms2m6A), 2-methylthio-N6-threonylcarbamoyladenosine (ms2t6A), 2-selenouridine (se2U), 2-thio-2′-O-methyluridine (s2Um), 2-thiocytidine (s2C), 2-thiouridine (s2U), 2′-O-methyladenosine (Am), 2′-O-methylcytidine (Cm), 2′-O-methylguanosine (Gm), 2′-O-methylinosine (Im), 2′-O-methylpseudouridine (Ym), 2′-O-methyluridine (Um), 2′-O-methyluridine 5-oxyacetic acid methyl ester (mcmo5Um), 2′-O-ribosyladenosine (phosphate) (Ar(p)), 2′-O-ribosylguanosine (phosphate) (Gr(p)), 3,2′-O-dimethyluridine (m3Um), 3-(3-amino-3-carboxypropyl)-5,6-dihydrouridine (acp3D), 3-(3-amino-3-carboxypropyl)pseudouridine (acp3Y), 3-(3-amino-3-carboxypropyl)uridine (acp3U), 3-methylcytidine (m3C), 3-methylpseudouridine (m3Y), 3-methyluridine (m3U), 4-demethylwyosine (imG-14), 4-thiouridine (s4U), 5,2′-O-dimethylcytidine (m5Cm), 5,2′-O-dimethyluridine (m5Um), 5-(carboxyhydroxymethyl)-2′-O-methyluridine methyl ester (mchm5Um), 5-(carboxyhydroxymethyl)uridine methyl ester (mchm5U), 5-(isopentenylaminomethyl)-2-thiouridine (inm5s2U), 5-(isopentenylaminomethyl)-2′-O-methyluridine (inm5Um), 5-(isopentenylaminomethyl)uridine (inm5U), 5-aminomethyl-2-geranylthiouridine (nm5ges2U), 5-aminomethyl-2-selenouridine (nm5se2U), 5-aminomethyl-2-thiouridine (nm5s2U), 5-aminomethyluridine (nm5U), 5-carbamoylhydroxymethyluridine (nchm5U), 5-carbamoylmethyl-2-thiouridine (ncm5s2U), 5-carbamoylmethyl-2′-O-methyluridine (ncm5Um), 5-carbamoylmethyluridine (ncm5U), 5-carboxyhydroxymethyluridine (chm5U), 5-carboxymethyl-2-thiouridine (cm5s2U), 5-carboxymethylaminomethyl-2-geranylthiouridine (cmnm5ges2U), 5-carboxymethylaminomethyl-2-selenouridine (cmnm5se2U), 5-carboxymethylaminomethyl-2-thiouridine (cmnm5s2U), 5-carboxymethylaminomethyl-2′-O-methyluridine (cmnm5Um), 5-carboxymethylaminomethyluridine (cmnm5U), 5-carboxymethyluridine (cm5U), 5-cyanomethyluridine (cnm5U), 5-formyl-2′-O-methylcytidine (f5Cm), 5-formylcytidine (f5C), 5-hydroxycytidine (ho5C), 5-hydroxymethylcytidine (hm5C), 5-hydroxyuridine (ho5U), 5-methoxycarbonylmethyl-2-thiouridine (mcm5s2U), 5-methoxycarbonylmethyl-2′-O-methyluridine (mcm5Um), 5-methoxycarbonylmethyluridine (mcm5U), 5-methoxyuridine (mo5U), 5-methyl-2-thiouridine (m5s2U), 5-methylaminomethyl-2-geranylthiouridine (mnm5ges2U), 5-methylaminomethyl-2-selenouridine (mnm5se2U), 5-methylaminomethyl-2-thiouridine (mnm5s2U), 5-methylaminomethyluridine (mnm5U), 5-methylcytidine (m5C), 5-methyldihydrouridine (m5D), 5-methyluridine (m5U), 5-taurinomethyl-2-thiouridine (tm5s2U), 5-taurinomethyluridine (tm5U), 7-aminocarboxypropyl-demethylwyosine (yW-86), 7-aminocarboxypropylwyosine (yW-72), 7-aminocarboxypropylwyosine methyl ester (yW-58), 7-aminomethyl-7-deazaguanosine (preQ1tRNA), 7-cyano-7-deazaguanosine (preQOtRNA), 7-methylguanosine (m7G), 8-methyladenosine (m8A), N2,2′-O-dimethylguanosine (m2Gm), N2,7,2′-O-trimethylguanosine (m2,7Gm), N2,7-dimethylguanosine (m2,7G), N2,N2,2′-O-trimethylguanosine (m2,2Gm), N2,N2,7-trimethylguanosine (m2,2,7G), N2,N2-dimethylguanosine (m2,2G), N2-methylguanosine (m2G), N4,2′-O-dimethylcytidine (m4Cm), N4,N4,2′-O-trimethylcytidine (m4,4Cm), N4,N4-dimethylcytidine (m4,4C), N4-acetyl-2′-O-methylcytidine (ac4Cm), N4-acetylcytidine (ac4C), N4-methylcytidine (m4C), N6,2′-O-dimethyladenosine (m6Am), N6,N6,2′-O-trimethyladenosine (m6,6Am), N6,N6-dimethyladenosine (m6,6A), N6-(cis-hydroxyisopentenyl)adenosine (io6A), N6-acetyladenosine (ac6A), N6-formyladenosine (f6A), N6-glycinylcarbamoyladenosine (g6A), N6-hydroxymethyladenosine (hm6A), N6-hydroxynorvalylcarbamoyladenosine (hn6A), N6-isopentenyladenosine (i6A), N6-methyl-N6-threonylcarbamoyladenosine (m6t6A), N6-methyladenosine (m6A), N6-threonylcarbamoyladenosine (t6A), Agmatidine (C+), Archaeosine (G+), cyclic N6-threonylcarbamoyladenosine (ct6A), dihydrouridine (D), epoxyqueuosine (oQtRNA), galactosyl-queuosine (galQtRNA), glutamyl-queuosine (gluQtRNA), hydroxy-N6-threonylcarbamoyladenosine (ht6A), hydroxywybutosine (OHyW), inosine (I), isowyosine (imG2), mannosyl-queuosine (manQtRNA), methylated undermodified hydroxywybutosine (OHyWy), methylwyosine (mimG), peroxywybutosine (o2yW), pseudouridine (Y), queuosine (QtRNA), undermodified hydroxywybutosine (OHyWx), uridine 5-oxyacetic acid (cmo5U), uridine 5-oxyacetic acid methyl ester (mcmo5U), wybutosine (yW), or wyosine (imG) modification.

In some embodiments, a nucleic acid agent provided and/or utilized in accordance with the present disclosure comprises a poly(A) element located 3′ to the complementary element and further comprises a 5′ cap element located 5′ to the complementary region. In some embodiments, a nucleic acid agent can comprise a poly(A) element located 3′ to the complementary element and further can comprise a 5′ cap element located 5′ to the complementary element. In some or all of the foregoing embodiments, the nucleic acid agent increases protein production from a target mRNA. In some such embodiments, the target mRNA is an mRNA that encodes a polypeptide whose expression is to be increased. In certain particular such embodiments, a target mRNA is an active allele of a gene associated with a disorder associated with a decrease in the expression of a protein from the mRNA. In some embodiments, the disorder is a haploinsufficiency disorder.

In some embodiments, a nucleic acid agent provided and/or utilized in accordance with the present disclosure is or comprises a nucleic acid molecule that comprises a poly(A) region located 3′ to the complementary region and further comprises a 5′ cap element located 5′ to the complementary region. In some embodiments, a nucleic acid agent provided and/or utilized in accordance with the present disclosure is or comprises a nucleic acid molecule that comprises a poly(A) element located 3′ to the complementary element, and further comprises a 5′ cap element located 5′ to the complementary region. In some or all of the foregoing embodiments, the nucleic acid agent increases protein production from a target mRNA. In some such embodiments, the target mRNA is an mRNA that encodes a polypeptide whose expression is to be increased. In certain particular such embodiments, a target mRNA is an active allele of a gene associated with a disorder associated with a decrease in the expression of a protein from the mRNA. In some embodiments, the disorder is a haploinsufficiency disorder.

### Encoding Agents

In some embodiments, a provided nucleic acid agent is or comprises one or more nucleic acid molecules that can be encoded by a nucleic acid sequence.

In certain particular embodiments, molecule nucleic acid agent useful in accordance with the present disclosure, and/or one or more components (e.g., nucleic acid molecules) thereof, is encoded by a sequence (e.g., in an encoding agent) that comprises or consists of SEQ ID NO: 1 or SEQ ID NO: 2, or a sequence that has at least 80%, at least 85%, at least 90%, at least 95%, or at least 99% sequence identity to SEQ ID NO: 2 or SEQ ID NO: 3.

plasmid for nucleic acid molecule with poly(A)
region located 3′ to the complementary region
(pJC1294)
SEQ ID NO: 2
CACCTAAATTGTAAGCGTTAATATTTTGTTAAAATTCGCGTTAAA
TTTTTGTTAAATCAGCTCATTTTTTAACCAATAGGCCGAAATCGG
CAAAATCCCTTATAAATCAAAAGAATAGACCGAGATAGGGTTGAG
TGTTGTTCCAGTTTGGAACAAGAGTCCACTATTAAAGAACGTGGA
CTCCAACGTCAAAGGGCGAAAAACCGTCTATCAGGGCGATGGCCC
ACTACGTGAACCATCACCCTAATCAAGTTTTTTGGGGTCGAGGTG
CCGTAAAGCACTAAATCGGAACCCTAAAGGGAGCCCCCGATTTAG
AGCTTGACGGGGAAAGCCGGCGAACGTGGCGAGAAAGGAAGGGAA
GAAAGCGAAAGGAGCGGGCGCTAGGGCGCTGGCAAGTGTAGCGGT
CACGCTGCGCGTAACCACCACACCCGCCGCGCTTAATGCGCCGCT
ACAGGGCGCGTCCCATTCGCCATTCAGGCTGCGCAACTGTTGGGA
AGGGCGATCGGTGCGGGCCTCTTCGCTATTACGCCAGCTGGCGAA
AGGGGGATGTGCTGCAAGGCGATTAAGTTGGGTAACGCCAGGGTT
TTCCCAGTCACGACGTTGTAAAACGACGGCCAGTGAATTGTAATA
CGACTCACTATAGGGCGAATTGGGTACCGGGCCCCCCCTCGAGGT
CGACGGTATCGATAAGCTTGATATCGAAATCGAACACAGGCTAAA
AATGTATATGCCCAAAGATAAAAAAAAAAAAAAAAAAAAAAAAAA
AAAAAAAAAAAAAAAAAAAAAAAATTCCTGCAGCCCGGGGGATCC
ACTAGTTCTAGAGCGGCCGCCACCGCGGTGGAGCTCCAGCTTTTG
TTCCCTTTAGTGAGGGTTAATTTCGAGCTTGGCGTAATCATGGTC
ATAGCTGTTTCCTGTGTGAAATTGTTATCCGCTCACAATTCCACA
CAACATACGAGCCGGAAGCATAAAGTGTAAAGCCTGGGGTGCCTA
ATGAGTGAGCTAACTCACATTAATTGCGTTGCGCTCACTGCCCGC
TTTCCAGTCGGGAAACCTGTCGTGCCAGCTGCATTAATGAATCGG
CCAACGCGCGGGGAGAGGCGGTTTGCGTATTGGGCGCTCTTCCGC
TTCCTCGCTCACTGACTCGCTGCGCTCGGTCGTTCGGCTGCGGCG
AGCGGTATCAGCTCACTCAAAGGCGGTAATACGGTTATCCACAGA
ATCAGGGGATAACGCAGGAAAGAACATGTGAGCAAAAGGCCAGCA
AAAGGCCAGGAACCGTAAAAAGGCCGCGTTGCTGGCGTTTTTCCA
TAGGCTCCGCCCCCCTGACGAGCATCACAAAAATCGACGCTCAAG
TCAGAGGTGGCGAAACCCGACAGGACTATAAAGATACCAGGCGTT
TCCCCCTGGAAGCTCCCTCGTGCGCTCTCCTGTTCCGACCCTGCC
GCTTACCGGATACCTGTCCGCCTTTCTCCCTTCGGGAAGCGTGGC
GCTTTCTCATAGCTCACGCTGTAGGTATCTCAGTTCGGTGTAGGT
CGTTCGCTCCAAGCTGGGCTGTGTGCACGAACCCCCCGTTCAGCC
CGACCGCTGCGCCTTATCCGGTAACTATCGTCTTGAGTCCAACCC
GGTAAGACACGACTTATCGCCACTGGCAGCAGCCACTGGTAACAG
GATTAGCAGAGCGAGGTATGTAGGCGGTGCTACAGAGTTCTTGAA
GTGGTGGCCTAACTACGGCTACACTAGAAGGACAGTATTTGGTAT
CTGCGCTCTGCTGAAGCCAGTTACCTTCGGAAAAAGAGTTGGTAG
CTCTTGATCCGGCAAACAAACCACCGCTGGTAGCGGTGGTTTTTT
TGTTTGCAAGCAGCAGATTACGCGCAGAAAAAAAGGATCTCAAGA
AGATCCTTTGATCTTTTCTACGGGGTCTGACGCTCAGTGGAACGA
AAACTCACGTTAAGGGATTTTGGTCATGAGATTATCAAAAAGGAT
CTTCACCTAGATCCTTTTAAATTAAAAATGAAGTTTTAAATCAAT
CTAAAGTATATATGAGTAAACTTGGTCTGACAGTTACCAATGCTT
AATCAGTGAGGCACCTATCTCAGCGATCTGTCTATTTCGTTCATC
CATAGTTGCCTGACTCCCCGTCGTGTAGATAACTACGATACGGGA
GGGCTTACCATCTGGCCCCAGTGCTGCAATGATACCGCGAGACCC
ACGCTCACCGGCTCCAGATTTATCAGCAATAAACCAGCCAGCCGG
AAGGGCCGAGCGCAGAAGTGGTCCTGCAACTTTATCCGCCTCCAT
CCAGTCTATTAATTGTTGCCGGGAAGCTAGAGTAAGTAGTTCGCC
AGTTAATAGTTTGCGCAACGTTGTTGCCATTGCTACAGGCATCGT
GGTGTCACGCTCGTCGTTTGGTATGGCTTCATTCAGCTCCGGTTC
CCAACGATCAAGGCGAGTTACATGATCCCCCATGTTGTGCAAAAA
AGCGGTTAGCTCCTTCGGTCCTCCGATCGTTGTCAGAAGTAAGTT
GGCCGCAGTGTTATCACTCATGGTTATGGCAGCACTGCATAATTC
TCTTACTGTCATGCCATCCGTAAGATGCTTTTCTGTGACTGGTGA
GTACTCAACCAAGTCATTCTGAGAATAGTGTATGCGGCGACCGAG
TTGCTCTTGCCCGGCGTCAATACGGGATAATACCGCGCCACATAG
CAGAACTTTAAAAGTGCTCATCATTGGAAAACGTTCTTCGGGGCG
AAAACTCTCAAGGATCTTACCGCTGTTGAGATCCAGTTCGATGTA
ACCCACTCGTGCACCCAACTGATCTTCAGCATCTTTTACTTTCAC
CAGCGTTTCTGGGTGAGCAAAAACAGGAAGGCAAAATGCCGCAAA
AAAGGGAATAAGGGCGACACGGAAATGTTGAATACTCATACTCTT
CCTTTTTCAATATTATTGAAGCATTTATCAGGGTTATTGTCTCAT
GAGCGGATACATATTTGAATGTATTTAGAAAAATAAACAAATAGG
GGTTCCGCGCACATTTCCCCGAAAAGTGC
plasmid for nucleic acid molecule with poly(A)
regions at both 3′ and 5′ end of the
complementary region (pJC1295)
SEQ ID NO: 3
CACCTAAATTGTAAGCGTTAATATTTTGTTAAAATTCGCGTTAAA
TTTTTGTTAAATCAGCTCATTTTTTAACCAATAGGCCGAAATCGG
CAAAATCCCTTATAAATCAAAAGAATAGACCGAGATAGGGTTGAG
TGTTGTTCCAGTTTGGAACAAGAGTCCACTATTAAAGAACGTGGA
CTCCAACGTCAAAGGGCGAAAAACCGTCTATCAGGGCGATGGCCC
ACTACGTGAACCATCACCCTAATCAAGTTTTTTGGGGTCGAGGTG
CCGTAAAGCACTAAATCGGAACCCTAAAGGGAGCCCCCGATTTAG
AGCTTGACGGGGAAAGCCGGCGAACGTGGCGAGAAAGGAAGGGAA
GAAAGCGAAAGGAGCGGGCGCTAGGGCGCTGGCAAGTGTAGCGGT
CACGCTGCGCGTAACCACCACACCCGCCGCGCTTAATGCGCCGCT
ACAGGGCGCGTCCCATTCGCCATTCAGGCTGCGCAACTGTTGGGA
AGGGCGATCGGTGCGGGCCTCTTCGCTATTACGCCAGCTGGCGAA
AGGGGGATGTGCTGCAAGGCGATTAAGTTGGGTAACGCCAGGGTT
TTCCCAGTCACGACGTTGTAAAACGACGGCCAGTGAATTGTAATA
CGACTCACTATAGGGCGAATTGGGTACCGGGCCCCCCCTCGAGGT
CGACGGTATCGATAAGCTTGATATCGAAAAAAAAAAAAAAAAAAA
AAAAAAAAAAAAAAAAAAAAAAAAAAAAAAAACCACAGGCTAAAA
ATGTATATGCCCAAAGATAAAAAAAAAAAAAAAAAAAAAAAAAAA
AAAAAAAAAAAAAAAAAAAAAATTCCTGCAGCCCGGGGGATCCAC
TAGTTCTAGAGCGGCCGCCACCGCGGTGGAGCTCCAGCTTTTGTT
CCCTTTAGTGAGGGTTAATTTCGAGCTTGGCGTAATCATGGTCAT
AGCTGTTTCCTGTGTGAAATTGTTATCCGCTCACAATTCCACACA
ACATACGAGCCGGAAGCATAAAGTGTAAAGCCTGGGGTGCCTAAT
GAGTGAGCTAACTCACATTAATTGCGTTGCGCTCACTGCCCGCTT
TCCAGTCGGGAAACCTGTCGTGCCAGCTGCATTAATGAATCGGCC
AACGCGCGGGGAGAGGCGGTTTGCGTATTGGGCGCTCTTCCGCTT
CCTCGCTCACTGACTCGCTGCGCTCGGTCGTTCGGCTGCGGCGAG
CGGTATCAGCTCACTCAAAGGCGGTAATACGGTTATCCACAGAAT
CAGGGGATAACGCAGGAAAGAACATGTGAGCAAAAGGCCAGCAAA
AGGCCAGGAACCGTAAAAAGGCCGCGTTGCTGGCGTTTTTCCATA
GGCTCCGCCCCCCTGACGAGCATCACAAAAATCGACGCTCAAGTC
AGAGGTGGCGAAACCCGACAGGACTATAAAGATACCAGGCGTTTC
CCCCTGGAAGCTCCCTCGTGCGCTCTCCTGTTCCGACCCTGCCGC
TTACCGGATACCTGTCCGCCTTTCTCCCTTCGGGAAGCGTGGCGC
TTTCTCATAGCTCACGCTGTAGGTATCTCAGTTCGGTGTAGGTCG
TTCGCTCCAAGCTGGGCTGTGTGCACGAACCCCCCGTTCAGCCCG
ACCGCTGCGCCTTATCCGGTAACTATCGTCTTGAGTCCAACCCGG
TAAGACACGACTTATCGCCACTGGCAGCAGCCACTGGTAACAGGA
TTAGCAGAGCGAGGTATGTAGGCGGTGCTACAGAGTTCTTGAAGT
GGTGGCCTAACTACGGCTACACTAGAAGGACAGTATTTGGTATCT
GCGCTCTGCTGAAGCCAGTTACCTTCGGAAAAAGAGTTGGTAGCT
CTTGATCCGGCAAACAAACCACCGCTGGTAGCGGTGGTTTTTTTG
TTTGCAAGCAGCAGATTACGCGCAGAAAAAAAGGATCTCAAGAAG
ATCCTTTGATCTTTTCTACGGGGTCTGACGCTCAGTGGAACGAAA
ACTCACGTTAAGGGATTTTGGTCATGAGATTATCAAAAAGGATCT
TCACCTAGATCCTTTTAAATTAAAAATGAAGTTTTAAATCAATCT
AAAGTATATATGAGTAAACTTGGTCTGACAGTTACCAATGCTTAA
TCAGTGAGGCACCTATCTCAGCGATCTGTCTATTTCGTTCATCCA
TAGTTGCCTGACTCCCCGTCGTGTAGATAACTACGATACGGGAGG
GCTTACCATCTGGCCCCAGTGCTGCAATGATACCGCGAGACCCAC
GCTCACCGGCTCCAGATTTATCAGCAATAAACCAGCCAGCCGGAA
GGGCCGAGCGCAGAAGTGGTCCTGCAACTTTATCCGCCTCCATCC
AGTCTATTAATTGTTGCCGGGAAGCTAGAGTAAGTAGTTCGCCAG
TTAATAGTTTGCGCAACGTTGTTGCCATTGCTACAGGCATCGTGG
TGTCACGCTCGTCGTTTGGTATGGCTTCATTCAGCTCCGGTTCCC
AACGATCAAGGCGAGTTACATGATCCCCCATGTTGTGCAAAAAAG
CGGTTAGCTCCTTCGGTCCTCCGATCGTTGTCAGAAGTAAGTTGG
CCGCAGTGTTATCACTCATGGTTATGGCAGCACTGCATAATTCTC
TTACTGTCATGCCATCCGTAAGATGCTTTTCTGTGACTGGTGAGT
ACTCAACCAAGTCATTCTGAGAATAGTGTATGCGGCGACCGAGTT
GCTCTTGCCCGGCGTCAATACGGGATAATACCGCGCCACATAGCA
GAACTTTAAAAGTGCTCATCATTGGAAAACGTTCTTCGGGGCGAA
AACTCTCAAGGATCTTACCGCTGTTGAGATCCAGTTCGATGTAAC
CCACTCGTGCACCCAACTGATCTTCAGCATCTTTTACTTTCACCA
GCGTTTCTGGGTGAGCAAAAACAGGAAGGCAAAATGCCGCAAAAA
AGGGAATAAGGGCGACACGGAAATGTTGAATACTCATACTCTTCC
TTTTTCAATATTATTGAAGCATTTATCAGGGTTATTGTCTCATGA
GCGGATACATATTTGAATGTATTTAGAAAAATAAACAAATAGGGG
TTCCGCGCACATTTCCCCGAAAAGTGC

In some embodiments, a nucleic acid agent provided and/or utilized in accordance with the present disclosure is or comprises (a) a nucleic acid molecule that is or includes a complementary element (e.g., a complementary region) that hybridizes with a target mRNA (i.e., with a target site therein) and; (b) a nucleic acid molecule that is or comprises a poly(A) element, wherein, in some embodiments, the complementary element and the poly(A) element are included in the same nucleic acid molecule (e.g., as regions thereof).

In some embodiments, a nucleic acid agent provided and/or utilized in accordance with the present disclosure can increase target mRNA expression. In some embodiments, a nucleic acid agent provided and/or utilized in accordance with the present disclosure can increase target mRNA in a subject, and/or in one or more cells or tissues (e.g., organs) thereof. In some embodiments, a nucleic acid agent provided and/or utilized in accordance with the present disclosure can increase target mRNA in a human (e.g., in one or more cells or tissues (e.g., organs) thereof). In some embodiments, a nucleic acid agent provided and/or utilized in accordance with the present disclosure can increase target mRNA in a mouse (e.g., in one or more cells or tissues (e.g., organs) thereof).

In some embodiments, a nucleic acid agent provided and/or utilized in accordance with the present disclosure can increase protein expression from a target mRNA. In some embodiments, a nucleic acid agent provided and/or utilized in accordance with the present disclosure can increase protein expression from a target mRNA in a subject. In some embodiments, a nucleic acid agent provided and/or utilized in accordance with the present disclosure can increase protein expression from a target mRNA in a human (e.g., in one or more cells or tissues (e.g., organs) thereof.

In some embodiments, a nucleic acid agent provided and/or utilized in accordance with the present disclosure can increase protein expression from a target mRNA in a mouse (e.g., in one or more cells or tissues thereof.

In some particular embodiments, a target mRNA encodes the MeCP2 protein. In some embodiments, a nucleic acid agent provided and/or utilized in accordance with the present disclosure comprises or consists of SEQ ID NO: 4 or 5, wherein the target mRNA encodes the MeCP2 protein in a human or mouse. In some embodiments, a nucleic acid agent provided and/or utilized in accordance with the present disclosure comprises or consists of a nucleic acid molecule that is, or that is or comprises one or more components of, SEQ ID NO: 6 or 7, wherein the target mRNA encodes the MeCP2 protein in a mouse.

In some embodiments, a target mRNA encodes the SYNGAP1 protein. In some embodiments, a nucleic acid agent provided and/or utilized in accordance with the present disclosure comprises or consists of a nucleic acid that is or includes one or more component of SEQ ID NO: 8 or 9, wherein the target mRNA encodes the SYNGAP1 protein in a human.

Mouse/Human pan Booster RNAs (G1-2tail)
SEQ ID NO: 4
AAAAAAAAAAAAAAAAAAAAAAAAAAAAAAAAAAAAAAAAAAAAA
AAAAAGAGCCCACTTTAAAACAAGCGCAGGTAAAAAAAAAAAAAA
AAAAAAAAAAAAAAAAAAAAAAAAAAAAAAAAAAA
Mouse/Human pan Booster RNAs (G1-1tail)
SEQ ID NO: 5
AAAAAAAAAAAAAAAAAAAAAAAAAAAAAAAAAAAAAAAAAAAAA
AAAAAGAGCCCACTTTAAAACAAGCGCAGGT
Mouse Specific Booster RNAs (G2-2tail)
SEQ ID NO: 6
AAAAAAAAAAAAAAAAAAAAAAAAAAAAAAAAAAAAAAAAAAAAA
AAAAAATATGGAAATACAGCACCAGCAGAAAAAAAAAAAAAAAAA
AAAAAAAAAAAAAAAAAAAAAAAAAAAAAAAA
Mouse Specific Booster RNAs (G2-1tail)
SEQ ID NO: 7
AAAAAAAAAAAAAAAAAAAAAAAAAAAAAAAAAAAAAAAAAAAAA
AAAAAATATGGAAATACAGCACCAGCAG
Human Syngap1 guide RNAs (Syng-tail)
SEQ ID NO: 8
AAAAAAAAAAAAAAAAAAAAAAAAAAAAAAAAAAAAAAAAAAAAA
AAAAACACCCTGAAGTTGAAAGTTTGGAGGTGCCA
Human Syngap1 guide RNAs (Syng-2tail)
SEQ ID NO: 9
AAAAAAAAAAAAAAAAAAAAAAAAAAAAAAAAAAAAAAAAAAAAA
AAAAACACCCTGAAGTTGAAAGTTTGGAGGTGCCAAAAAAAAAAA
AAAAAAAAAAAAAAAAAAAAAAAAAAAAAAAAAAAAAAAA

In some embodiments, provided herein are recombinant expression systems that include a nucleic acid sequence encoding a nucleic acid molecule comprising one or both of (i) a complementary element that hybridizes with a target mRNA and (ii) a poly(A) element.

In some embodiments, provided herein are expression vectors that include a recombinant expression system described herein. In some embodiments, an expression vector is a viral vector.

In some embodiments, a viral vector is an adeno-associated viral vector (AAV), a lentiviral vector, or an adenoviral vector.

In some embodiments, provided herein are pharmaceutical compositions that comprise or deliver (e.g., by expression) a nucleic acid agent, or element thereof (e.g., a nucleic acid molecule, such as an RNA molecule, that is or comprises one or both of i) a complementary element that hybridizes with a target mRNA (i.e., with a site therein) and (ii) a poly(A) element. In some embodiments, a provided pharmaceutical composition comprises or delivers a recombinant expression system (e.g., an expression vector) as described herein.

### Therapeutic Applications

Provided herein are technologies for increasing translation of a target mRNA. Thus, in some embodiments, provided are technologies for increasing level of a polypeptide of interest encoded by such target mRNA.

Those skilled in the art, reading the present disclosure will appreciate a variety of applications, including a variety of therapeutic applications, provided thereby.

In some embodiments, provided technologies achieve increased expression of a polypeptide beneficial in the treatment of a disorder in a subject, for example by administering to the subject a therapeutically effective amount of a pharmaceutical composition described herein (e.g., that comprises or delivers a nucleic acid agent as described herein).

In some embodiments, a provided method of treatment comprises a step of administering to a subject a composition that comprises or delivers a nucleic acid agent whose complementary element hybridizes with a target mRNA encoding an endogenous protein. In some embodiments, a provided method of treatment comprises a step of administering to a subject a composition that comprises or delivers a nucleic acid agent whose complementary element hybridizes with a target mRNA encoding an exogenous protein.

In some embodiments, increased protein expression achieved in accordance with the present disclosure provides a protein that is beneficial to a cell or tissue (e.g., organ) of interest in a subject. In some embodiments, increased protein expression achieved in accordance with the present disclosure provides a protein that is detrimental to an undesirable cell or tissue (e.g., organ) in a subject. To give but one such example, in some embodiments, provided technologies achieve and/or increase expression of a protein that is toxic to (e.g., that is a suicide protein) in an undesirable cell such as, for example a cancer cell and/or a cell therapy product (e.g., an engineered cell).

In some embodiments, provided herein are methods of treating or preventing a disorder in a subject by administering any one of the recombinant expression systems or any one of the expression vectors described herein to the subject.

In some particular embodiments, a treated disorder is a haploinsufficiency disorder. In some embodiments, a treated disorder is a cancer. In some embodiments, a cancer is a B-ALL tumor, liver cancer, or breast cancer.

In some embodiments, provided herein are compositions that comprise and/or deliver a nucleic acid agent (e.g., a nucleic acid molecule) molecule for use in increasing protein expression from a target mRNA. In some embodiments, provided herein are compositions that comprise and/or deliver a nucleic acid agent (e.g., a nucleic acid molecule) for use in increasing target mRNA expression. In some embodiments, such a nucleic acid agent (e.g., nucleic acid molecule) includes a poly(A) element located 5′ to the complementary element. In some embodiments, such a nucleic acid agent (e.g., nucleic acid molecule) includes a poly(A) element located 3′ to the complementary element. In some embodiments, such a nucleic acid agent (e.g., nucleic acid molecule) includes a poly(A) element located 3′ to the complementary element and wherein further comprises a 5′ cap element located 5′ to the complementary element. In some embodiments, such a nucleic acid agent (e.g., nucleic acid molecule) comprises a first poly(A) element located 5′ to the complementary element and a second poly(A) element located 3′ to the complementary element. In some embodiments, such a nucleic acid agent (e.g. nucleic acid molecule) can comprises a poly(A) element located 3′ to the complementary element and a second poly(A) element located 5′ to the complementary element, wherein the nucleic acid molecule increases protein expression from a target mRNA. In some embodiments, such a nucleic acid agent is or comprises a nucleic acid molecule (e.g., an RNA molecule) which, for example, can comprise a poly(A) element located 3′ to the complementary element and, in some embodiments, can further comprise a 5′ cap element located 5′ to the complementary element, wherein the nucleic acid molecule increases protein expression from a target mRNA. In some embodiments, such a nucleic acid agent (e.g. nucleic acid molecule includes a poly(A) element located 5′ to the complementary element, wherein the nucleic acid agent increases a target mRNA expression. In some embodiments, such a nucleic acid agent (e.g. nucleic acid molecule) includes a poly(A) element located 3′ to the complementary element, wherein the nucleic acid agent increases a target mRNA expression. In some embodiments, such a nucleic acid agent (e.g. nucleic acid molecule) includes a poly(A) element located 5′ to the complementary element and a second poly(A) element located 3′ to the complementary element, wherein the nucleic acid agent increases a target mRNA expression. In some particular such embodiments, a target mRNA is an mRNA of an active allele of a gene associated with a disorder associated with a decrease in the expression of a protein from the mRNA. In some embodiments, the disorder is a haploinsufficiency disorder.

In some embodiments, provided herein are compositions that comprise and/or deliver a nucleic acid agent as described herein for use in treating haploinsufficiency disorders. In some embodiments, provided herein are methods of use of provided nucleic acid agents (e.g., nucleic acid molecules) for treating haploinsufficiency disorders. In some embodiments, provided herein are methods of use of the nucleic acid agents (e.g., nucleic acid molecules) in the manufacture of a medicament for treating haploinsufficiency disorders.

Haploinsufficiency occurs when one gene allele is inactivated and the amount of gene product expressed from the remaining active allele is insufficient for proper gene function. A number of disorders are associated with, or are caused by haploinsufficiency.

In some embodiments, a haploinsufficiency disorder is selected from 5qsyndrome, Adams-Oliver syndrome 1, Adams-Oliver syndrome 3, Adams-Oliver syndrome 5, Adams-Oliver syndrome 6, Alagille syndrome 1, Autoimmune lymphoproliferative syndrome type IA, Autoimmune lymphoproliferative syndrome type V, Autosomal dominant deafness-2A, Brain malformations with or without urinary tract defects (BRMUTD), Carney complex type 1, CHARGE syndrome, Cleidocranial dysplasia, Currarino syndrome, Denys-Drash syndrome/Frasier syndrome, Developmental delay, intellectual disability, obesity, and dysmorphic features (DIDOD), DiGeorge syndrome (TBXI-associated), Dravet syndrome, Duane-radial raysyndrome, Ehlers-Danlos syndrome (classic-like), Ehlers-Danlos syndrome (vascular type), Feingold syndrome 1, Frontotemporal lobar degeneration with TDP43 inclusions (FTLD-TDP), GRN-related, GLUT I deficiency syndrome, Greig cephalopolysyndactyly syndrome, Hereditary hemorrhagic telangiectasia type 1, Holoprosencephaly 3, Holoprosencephaly 4, Holoprosencephaly 5, Holt-Oram syndrome, Hypoparathyroidism, sensorineural deafness, andrenal disease (HDR), Kleefstra syndrome 1, Klippel-Trenaunay syndrome (AAGF-related), Leri-Weill dyschondrosteosis, Marfan syndrome, Mental retardation and distinctive facial features with or without cardiac defects (MRFACD), Mental retardation, autosomal dominant 1, Mental retardation, autosomal dominant 19, Mental retardation, autosomal dominant 29, Nail-patella syndrome (NPS), Phelan-McDermid syndrome, Pitt-Hopkins syndrome, Primary pulmonary hypertension 1, Rett syndrome (congenital variant), Smith-Magenis syndrome (RAII associated), Sotos syndrome 1, Sotos syndrome 2, Stickler syndrome type I, Supravalvular aorticstenosis, SYNGAPI-related intellectual disability, Treacher Collins syndrome, Trichorhinophalangeal syndrome type I, Ulnar-mammary syndrome, van der Woude syndrome1, Waardenburg syndrome type 1, Waardenburg syndrome type 2A, and Waardenburg syndrometype 4C.

In some embodiments, ahaploinsufficient gene is selected from the group consisting of AGGFI, ARHGAP31, BMPR2, CHD7, COL2Al, COL3Al, CTLA4, CTNNBI, DLL4, EHMTI, ELN, ENG, FAS, FBNI, FOXGI, GATA3, GLI3, GRN, IRF6, JAGI, KCNQ4, LMXIB, MBD5, MED13L, MITF, MNX, MYCN, NFIA, NFIX NOTCH1, NSDI, PAX3, PHIP, PRKARIA, RAil, RBPJ, RPS14, RUNX2, SALL4, SCNIA, SETBPI, SHANK3, SHH, SHOX, SLC2Al/GLUT1, SOXI0, SYNGAPI, TBXI, TBX3, TBX5, TCF4, TCOFI, TGIFI, TNB, TRPSI, WTI, ZIC2, and combinations thereof. In some embodiments, a haploinsufficient gene can be the CTNNBI gene. In some embodiments, a haploinsufficient gene can be the PURA gene.

In some embodiments, a target mRNA is an mRNA of an active allele of a tumor suppressor gene, for example associated with a haploinsufficiency disorder. In some embodiments, a tumor suppressor gene is selected from the group consisting of CD19, CD22, C/EBP-alpha, HER2, CDKN1A, TP53, VHL, CEBPA, INTS6, HIC1, PTEN, CDH1, VEZT, CPYSL3, NKX3-1, PAWR, sFHIT, DIRAS1, KLF4, WT1, and MAS1.

In some embodiments, a haploinsufficiency disorder and haploinsufficient gene combination is a combination shown in Table 1.

TABLE 1
Haploinsufficiency disorders and genes.

| | Haploinsufficient |
|---|---|
| Haploinsufficiency Disorder | Gene |
| 5q-syndrome | RPS14 |
| Adams-Oliver syndrome I | ARHGAP31 |
| Adams-Oliver syndrome 3 | RBPJ |
| Adams-Oliver syndrome 5 | NOTCH1 |
| Adams-Oliver syndrome 6 | DLL4 |
| Alagille syndrome I | JAG1, NOTCH2 |
| Autoimmune lymphoproliferative syndrome type IA | FAS |
| Autoimmune lymphoproliferative syndrome type V | CTLA4 |
| Autosomal dominant deafness-2A | KCNQ4 |
| Brain malformations with or without urinary tract | NFIA |
| defects (BRMUTD) | |
| Carney complex type 1 | PRKARlA |
| CHARGE syndrome | CHD7 |
| Cleidocranial dysplasia | RUNX2 |
| Congenital muscular dystrophy type 1A | LAMA-2 |
| Currarino syndrome | MNXl |
| Cystic fibrosis | CFTR |
| Denys-Drash syndrome/Frasier syndrome | WTl |
| Developmental delay, intellectual | PHIP |
| disability, obesity, and dysmorphic features (DIDOD) | |
| DiGeorge syndrome (TBXl-associated) | TBXl |
| Dravet syndrome | SCNlA |
| Duane-radial ray syndrome | SALL4 |
| Duchenne Muscular Dystrophy | Dystrophin |
| Ehlers-Danlos syndrome (classic-like) | TNXB |
| Ehlers Danlos syndrome (vascular type) | COL3Al |
| Feingold syndrome 1 | MYCN |
| Frontotemporal lobar degeneration with TDP43 | GRN |
| inclusions (FTLD-TDP), GRN-related | |
| GLUT1 deficiency syndrome | SLC2Al |
| Greig cephalopolysyndacty1y syndrome | GLI3 |
| Hereditary hemorrhagic telangiectasia type 1 | ENG |
| Holoprosencephaly 3 | SHH |
| Holoprosencephaly 4 | TGIFl |
| Holoprosencephaly 5 | ZIC2 |
| Holt-Oram syndrome | TBX5 |
| Hypoparathyroidism, sensorineural deafness, and | GATA3 |
| renal disease (HDR) | |
| Kleefstra syndrome 1 | EHMTl |
| Klippel-Trenaunay syndrome (AAGF-related) | AGGFl |
| Leri-Weill dyschondrosteosis | SHOX |
| Marfan syndrome | FBNl |
| Mental retardation and distinctive facial features | MED13L |
| with or without cardiac defects (MRFACD) | |
| Mental retardation, autosomal dominant 1 | MBD5 |
| Mental retardation, autosomal dominant 19 | CTNNBl |
| Mental retardation, autosomal dominant 29 | SETBPl |
| Nail-patella syndrome (NPS) | LMXlB |
| Phelan-McDermid syndrome | SHANK3 |
| Pitt-Hopkins syndrome | TCF4 |
| Primary pulmonary hypertension 1 | BMPR2 |
| Rett syndrome (congenital variant) | FOXGl |
| Smith-Magenis syndrome (RAIl-associated) | RAI1 |
| Sotos syndrome 1 | NSDl |
| Sotos syndrome 2 | NFIX |
| Stickler syndrome type I | COL2Al |
| Supravalvular aortic stenosis | ELN |
| SYNGAPl-related intellectual disability | SYNGAPl |
| Treacher Collins syndrome | TCOFl |
| Trichorhinophalangeal syndrome type I | TRPSl |
| Ulnar-mammary syndrome | TBX3 |
| van der Woude syndrome 1 | IRF6 |
| Waardenburg syndrome type 1 | PAX3 |
| Waardenburg syndrome type 2A | MITF |
| Waardenburg syndrome type 4C | SOXlO |
| Wilsons disease | ATP7B |

### CNS Haploinsufficiency Disorders

In some embodiments, a haploinsufficiency disorder is a CNS haploinsufficiency disorder. In some embodiments, a haploinsufficiency disorder is selected from the group consisting of episodic ataxia, familial hemiplegia migraine, CDKL5 deficiency disorder, CHD2 myoclonic encephalopathy, familial focal epilepsy with variable loci, FOXG1 syndrome, benign familial neonatal seizures, Rett syndrome, Dravat syndrome, SCN2A-epileptic encephalopathy, SCN2A-developmental encephalopathy, SCN8A-epileptic encephalopathy, SC8A familial infantile epilepsy, early infantile epileptic encephalopathy, myoclonic-atonic epilepsy, early infantile epileptic encephalopathy, SYNGAPI-related intellectual disability, tuberous sclerosis, Lennox-Gastaut Syndrome, FoxG1 syndrome, KCNQ2-related epileptic encephalopathy, PCDH19-related epilepsy, SLC6A1-related myoclonic-astatic epilepsy, STXBP1-related epileptic encephalopathy, SYNGAPI syndrome, and combinations thereof.

In some embodiments, a haploinsufficiency gene is selected from the group consisting of SCNIA, SCN2A, SCN8A, SCN12A5, SPTAN1, CDKL5, CHD2, FOXG1, KCNQ2, PCDH19, SLC6A1, STBP1, SYNGAP1, CACNA1A, DEPDC5, MECP2, TSC1, TSC2, and combinations thereof.

In some embodiments, a haploinsufficiency disorder and haploinsufficient gene combination is a combination shown in Table 2

TABLE 2
CNS haploinsufficiency disorders and genes

| Haploinsufficiency Disorder | Haploinsufficient Gene |
|---|---|
| Episodic ataxia, Familial hemiplegia migraine | CACNA1A |
| CDKL5 deficiency disorder | CDKL5 |
| CHD2 myoclonic encephalopathy | CHD2 |
| Lennox-Gastaut Syndrome | CHD2 |
| Familial focal epilepsy with variable loci | DEPDC5 |
| FOXG1 syndrome | FOXG1 |
| Benign familial neonatal seizures | KCNQ2 |
| Rett syndrome | MECP2 |
| Dravet syndrome | SCN1A |
| SCN2A-epileptic encephalopathy | SCN2A |
| SCN2A-developmental encephalopathy | SCN2A |
| SCN8A-epileptic encephalopathy | SCN8A |
| SCN8A familial infantile epilepsy | SCN8A |
| Early infantile epileptic encephalopathy | SLC12A5 |
| Myoclonic-atonic epilepsy | SLC6A1 |
| Early infantile epileptic encephalopathy | SPTAN1 |
| SYNGAP1-related intellectual disability | SYNGAP1 |
| Tuberous sclerosis | TSC1 |
| Tuberous sclerosis | TSC2 |
| KCNQ2-related epileptic encephalopathy | KCNQ2 |
| SLC6A1-related myoclonic-astatic epilepsy | SLC6A1 |
| STXBP1-related epileptic encephalopathy | STXBP1 |
| SYNGAP1 syndrome | SYNGAP1 |

### EXAMPLES

The disclosure is further described in the following examples, which do not limit the scope of the disclosure described in the claims.

### Example 1—Construct Preparation and In-Vitro MeCP2 mRNA Synthesis

To prepare poly(A) region+short RNA construct to target MeCP2 3′UTR, the optimal short RNA targeting 3′UTR of MeCP2 (CCACAGGCTAAAAATGTATATGCCCAAAGA (SEQ ID NO: 1)) was screened and selected, and 50 adenosine (A) nucleotides were added either to the 3′ or 5′ end of the sequence (FIGS. 1A-1C). Oligos with different combinations of polyA regions and short targeting RNA was designed and synthesized by IDT (Integrated DNA Technologies). The synthesized oligos as a gene block cloned in pJc315 (bluescript-SK+) using the Gibson assembly strategy. The constructs with one polyA region in the 3′ of the sequence (pJC1294) and two poly A regions in both 3′ and 5′ ends (pJC1295) were chosen for in vitro transcription (IVT assay) and further investigations. For IVT, pJc1294 and pJC1295 digested with SacI restriction enzyme and gel purified. The linearized plasmids were transcribed using the HiScribe T7 High Yield RNA Synthesis Kit (New England Biolab inc.) according to the manufacture protocol. The RNA product was purified using Monarch RNA cleanup kit (New England Biolab inc.) for cell transfection.

### Example 2—Cell Preparation and RNA Transfection

Human embryonic kidney 293 cells line were grown in Dulbecco's Modified Eagle's Medium (DMEM) with 10% FBS. Cells were incubated at 37° C. in 5% CO2 and their medium was changed every 48 to 72 hours. To transfect cells with small mRNAs, Hek-293 cells at about %70 confluency were split 1; 2 the day before transfection. Immediately prior to transfection, cells were trypsinized, pelleted and resuspended in the fresh media and diluted to 500000 cells/mL. The cell suspension was transfected with TransIT®-mRNA Transfection Kit (Mirus) according to the manufacture instruction. Transfection complexes added to the cell suspension in 12 well plates and incubated at 37° C. in 5% CO2 for 6 hours. The whole cell lysate and total RNA extracted using RIPA buffer and Trizol/chloroform respectively.

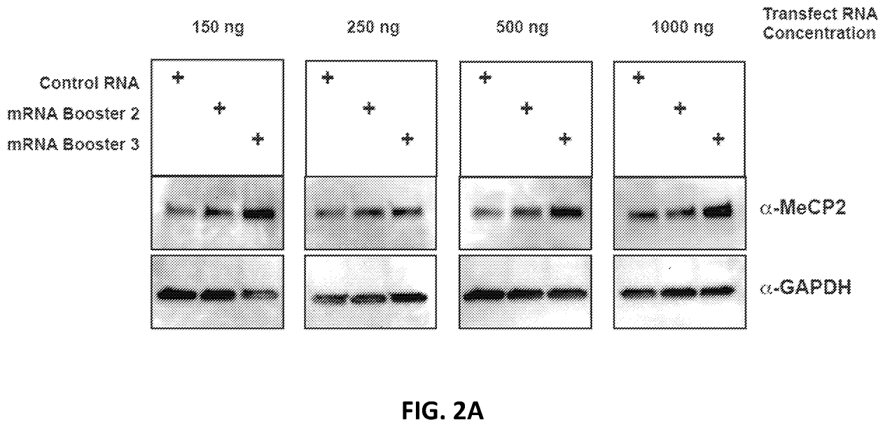

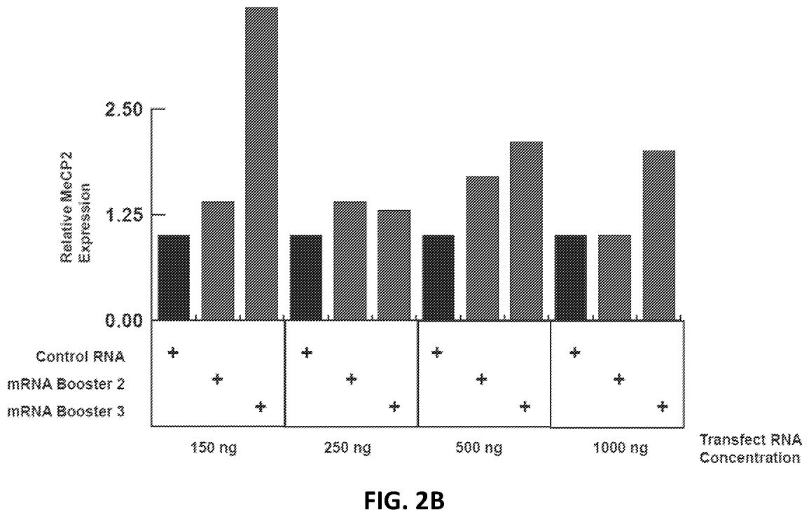

Western blot analysis was performed on the whole cell extract and the MeCP2 protein level was shown using 1:1000 dilution of MeCP2 (D4F3) XP® Rabbit mAb 3456 cell signaling antibody. Obtained results were quantified and normalized with the level of GAPDH protein (FIGS. 2A-2B).

The cDNA synthesis proceeded for the RNA extract and qRT-PCR performed using the PowerUp SYBER Green Master Mix (applied biosystem) and designed primer set for MeCP2 mRNA.

### Example 3—Lipid Nanoparticle (LNP) Synthesis and Characterization and In Vivo mRNA Synthesis

The Booster RNAs compatible with 3′UTR of MeCP2 mRNA in mouse have been designed according to the style suggested for mRNA Booster 3 (FIG. 1C) and cloned in T7 promoter plasmid for IVT. The 5′modified synthesized Booster RNAs were sent for packaging into LNPs for in vivo injection.

LNPs were synthesized by directly adding an organic phase containing the lipids to an aqueous phase containing the RNAs in a 1.5-ml microcentrifuge tubes. To prepare the organic phase, a mixture of Dlin-MC3 DMA, cholesterol (Sigma-Aldrich), DMG-PEG2000 (Avanti), and the helper lipid (18PG) (Avanti) were solubilized in ethanol. To prepare the aqueous phase, corresponding RNA was prepared in 25 mM magnesium acetate buffer (pH 4.0, Fisher). The storage temperature for all RNAs was −80° C., and all RNAs were allowed to thaw on ice before use. For larger scale LNP production, the aqueous and ethanol phases prepared were mixed at a 3:1 ratio in an FNC device using syringe pumps. Resultant LNPs were dialyzed against DI water in a 100,000 MWCO cassette (Fisher) at 4° C. for 24 h and were stored at 4° C. before injection.

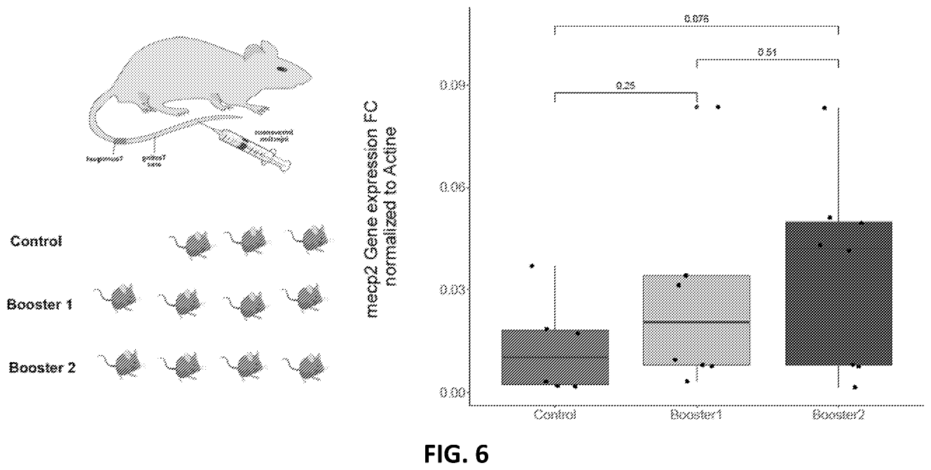

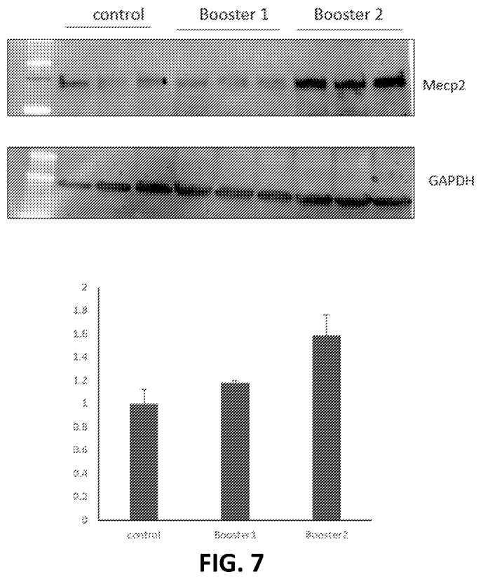

The LNPs were given through i.v. injection at a predetermined dose per mouse. (25 μg RNA per mice was used). After 24 h, the mice were scarified, and the liver tissue was isolated (FIGS. 6-7).

### Example 4—Construct Preparation and In-Vitro CTNNB1 and PurA mRNA Synthesis

Booster RNAs targeting 3′UTR of CTNNB1 (β-catenin) and Pur-α genes were prepared as described in Example 1, wherein optimal short RNAs with highest guiding score were selected and 50 adenosine(A) nucleotides were added to both 5′ and 3′ ends of the sequence. The 5′ end was also modified with cap analog to prevent the RNA digestion (FIG. 1C). Oligos were designed and synthesized by IDT (Integrated DNA Technologies). The synthesized oligos were used as a gene block cloned in pJc315 (bluescript-SK+) using the Gibson assembly strategy and used as a template for IVT (following the protocol described herein).

Human embryonic kidney 293 cells line were grown in Dulbecco's Modified Eagle's Medium (DMEM) with 10% FBS. Cells were incubated at 37° C. in 5% CO2 and their medium was changed every 48 to 72 hours. To transfect cells with small mRNAs, Hek-293 cells at about %70 confluency were split 1; 2 the day before transfection. Immediately prior to transfection, cells were trypsinized, pelleted and resuspended in the fresh media and diluted to 500000 cells/mL. The cell suspension was transfected with TransIT®-mRNA Transfection Kit (Mirus) according to the manufacture instruction. Transfection complexes added to the cell suspension in 12 well plates and incubated at 37° C. in 5% CO2 for 6 hours. The whole cell lysate and total RNA extracted using RIPA buffer and Trizol/chloroform respectively.

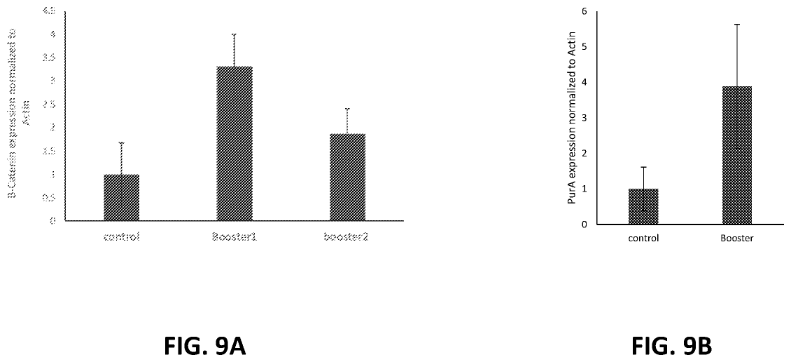

The human embryonic kidney 293 cells line were grown and transfected with Booster RNA overnight and cells were harvested next day for RNA extraction and qPCR. The result of qPCR indicated there is up to 3-fold increase in the level of either of CTNNB1 or PurA mRNA when they were exposed to Booster RNA overnight in culture (FIGS. 9A-9B).

### Example 5—Deep Brain Perfusion Experiments: 24 Hours and 48 Hours

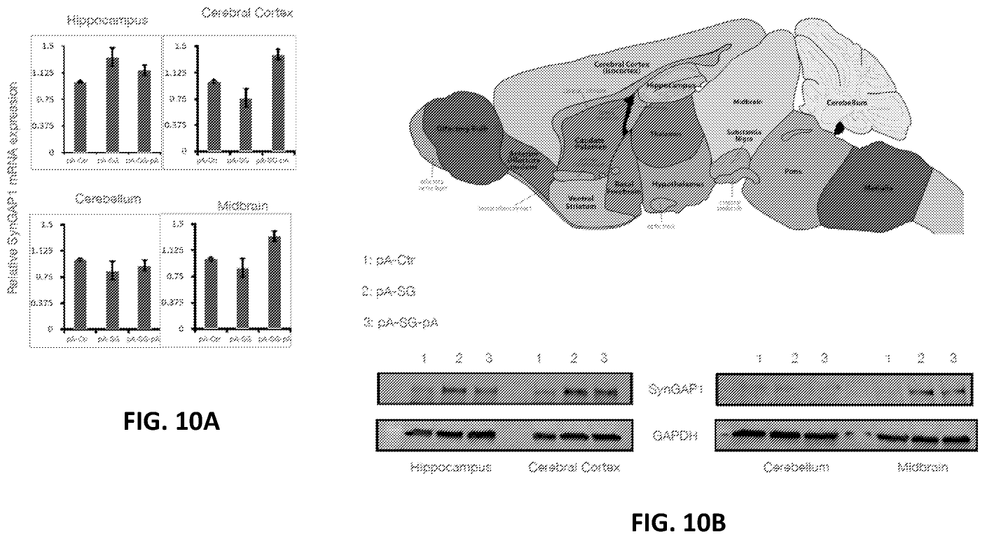

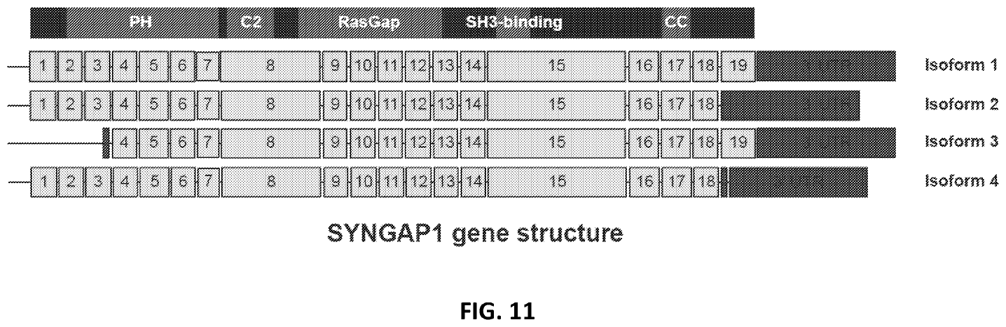

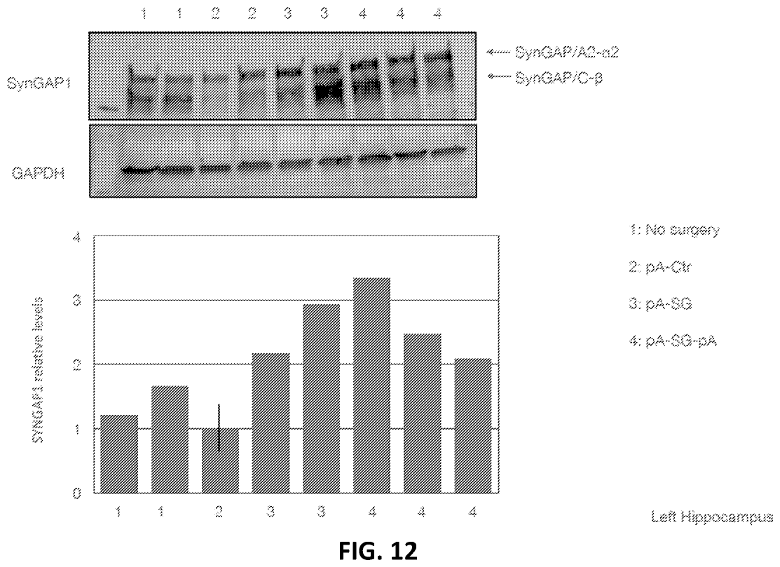

The most effective booster across the 3′UTR of the SYNGAPI to enhance the protein expression was selected. Two different designs of booster RNAs PA-SG and PA-SG-PA have been selected for in vivo study. Using the in vitro transcription (IVT) technology, designed booster were synthesized and purified, then encapsulated into the LNPs. The technology of deep brain perfusion was used to deliver LNPs into specific mouse brain regions and investigate its efficacy in vivo. The technology involves a titanium catheter that is placed into the curved aspect of the hippocampus and enables repeated localized injection without repeated surgical procedure. Wild type animals were injected with 25 micrograms of the booster RNA alongside the RNA scramble as a control. The experiment was repeated two times. In the first attempt, injected animals were sacrificed 24 hours after injection (FIGS. 10A-10B) and in the second experiment, animals were sacrificed 48 hours post injection (FIG. 12). The whole brain was dissected into the four major regions; hippocampus, cortex, midbrain and cerebellum, which were analyzed separately. The result of both experiments indicated enhanced Syngap1 in both protein and RNA level.

What is claimed is:

1. A nucleic acid agent comprising:
(a) a complementary element that hybridizes with a target mRNA; and
(b) a poly(A) element.

2. The nucleic acid element of claim 1, wherein the complementary element hybridizes to a 3′ untranslated element of the target mRNA.

3. The nucleic acid agent of claim 1 or 2, wherein the complementary element comprises RNA or DNA.

4. The nucleic acid agent of any one of claims 1-3, wherein the poly(A) element is located 5′ to the complementary element.

5. The nucleic acid agent of any one of claims 1-4, wherein the poly(A) element is located 3′ to the complementary element.

6. The nucleic acid agent of claim 5, wherein the poly(A) element is located 3′ to the complementary element and the nucleic acid agent further comprises a 5′ cap element located 5′ to the complementary element.

7. The nucleic acid agent of any one of claims 1-6, wherein the nucleic acid agent comprises a first poly(A) element located 5′ to the complementary element and a second poly(A) element located 3′ to the complementary element.

8. The nucleic acid agent of any one of claims 1-7, wherein the poly(A) element comprises about 20 nucleotides.

9. The nucleic acid agent of any one of claims 1-7, wherein the poly(A) element comprises about 50 nucleotides.

10. The nucleic acid agent of any one of claims 1-7, wherein the poly(A) element comprises about 75 nucleotides.

11. The nucleic acid agent of any one of claims 1-10, wherein the nucleic acid agent further comprises a nucleotide modification.

12. The nucleic acid agent of claim 11, wherein the nucleotide modification is a N¹-methyladenosine (m¹A), N⁶-methyladenosine (m⁶A), or adenosine to inosine (A-to-I) modification.

13. The nucleic acid agent of claim 11, wherein the nucleotide modification is a pseudouracil, M1-pseudouracil, 5-methoxyuridine (5moU), or N4-acetylcytidine modification.

14. The nucleic acid agent of any one of claims 1-13, wherein the nucleic acid agent is encoded by a sequence that comprises or consists of SEQ ID NO: 2 or SEQ ID NO: 3, or a sequence that has at least 80%, at least 85%, at least 90%, at least 95%, or at least 99% sequence identity to SEQ ID NO: 2 or SEQ ID NO: 3.

15. The nucleic acid agent of any one of claims 1-14, wherein the target mRNA is an mRNA of an active allele of a gene associated with a disorder associated with a decrease in the expression of a protein from the mRNA.

16. The nucleic acid agent of claim 15, wherein the disorder is a haploinsufficiency disorder.

17. The nucleic acid agent of claim 15 or 16, wherein the target mRNA encodes the MeCP2 protein.

18. The nucleic acid agent of claim 17, wherein the complementary element comprises or consists of SEQ ID NO: 1.

19. A recombinant expression system comprising a nucleic acid sequence encoding a nucleic acid agent comprising (i) a complementary element that hybridizes with a target mRNA and (ii) a poly(A) element.

20. The recombinant expression system of claim 19, wherein the complementary element hybridizes to a 3′ untranslated element of the target mRNA.

21. The recombinant expression system of claim 19 or 20, wherein the complementary element comprises RNA or DNA.

22. The recombinant expression system of any one of claims 19-21, wherein the poly(A) element is located 5′ to the complementary element.

23. The recombinant expression system of any one of claims 19-22, wherein the poly(A) element is located 3′ to the complementary element.

24. The recombinant expression system of claim 23, wherein the poly(A) element is located 3′ to the complementary element and wherein the RNA molecule further comprises a 5′ cap element located 5′ to the complementary element.

25. The recombinant expression system of any one of claims 19-24, the nucleic acid molecule comprises a first poly(A) element located 5′ to the complementary element and a second poly(A) element located 3′ to the complementary element.

26. The recombinant expression system of any one of claims 19-25, wherein the poly(A) element comprises about 20 nucleotides.

27. The recombinant expression system of any one of claims 19-25, wherein the poly(A) element comprises about 50 nucleotides.

28. The recombinant expression system of any one of claims 19-25, wherein the poly(A) element comprises about 75 nucleotides.

29. The recombinant expression system of any one of claims 19-28, wherein the nucleic acid agent further comprises a nucleotide modification.

30. The recombinant expression system of claim 29, wherein the nucleotide modification is a N¹-methyladenosine (m¹A), N⁶-methyladenosine (m⁶A), or adenosine to inosine (A-to-I) modification.

31. The recombinant expression system of claim 29, wherein the nucleotide modification is a pseudouracil, M1-pseudouracil, 5-methoxyuridine (5moU), or N4-acetylcytidine modification.

32. The recombinant expression system of any one of claims 19-31, wherein the recombinant expression system is encoded by a sequence that comprises or consists of SEQ ID NO: 2 or SEQ ID NO: 3, or a sequence that has at least 80%, at least 85%, at least 90%, at least 95%, or at least 99% sequence identity to SEQ ID NO: 2 or SEQ ID NO: 3.

33. The recombinant expression system of any one of claims 19-32, wherein the target mRNA is an mRNA of an active allele of a gene associated with a disorder associated with a decrease in the expression of a protein from the mRNA.

34. The recombinant expression system of claim 33, wherein the disorder is a haploinsufficiency disorder.

35. The recombinant expression system of claim 33 or 34, wherein the target mRNA encodes the MeCP2 protein.

36. The recombinant expression system claim 35, wherein the nucleic acid agent is or comprises a nucleic acid molecule that comprises or consists of SEQ ID NO: 1.

37. An expression vector comprising the recombinant expression system of any one of claims 19-36.

38. The expression vector of claim 37, wherein the expression vector is a viral vector.

39. The expression vector of claim 38, wherein the viral vector is an adeno-associated viral vector (AAV), a lentiviral vector, or an adenoviral vector.

40. A pharmaceutical composition that comprises or delivers the nucleic acid agent of any one of claims 1-18, the recombinant expression system of any one of claims 19-36, or the expression vector of any one of claims 37-39.

41. A method of treating a haploinsufficiency disorder in a subject, the method comprising: administering to the subject a therapeutically effective amount of the pharmaceutical composition of claim 40.

42. The method of claim 41, wherein the therapeutically effective amount of the pharmaceutical composition enhances the target mRNA protein expression.

43. The method of claim 41 or 42, wherein the haploinsufficiency disorder is selected from the group consisting from 5qsyndrome, Adams-Oliver syndrome 1, Adams-Oliver syndrome 3, Adams-Oliver syndrome 5, Adams-Oliver syndrome 6, Alagille syndrome 1, Autoimmune lymphoproliferative syndrome type IA, Autoimmune lymphoproliferative syndrome type V, Autosomal dominant deafness-2A, Brain malformations with or without urinary tract defects (BRMUTD), Carney complex type 1, CHARGE syndrome, Cleidocranial dysplasia, Currarino syndrome, Denys-Drash syndrome/Frasier syndrome, Developmental delay, intellectual disability, obesity, and dysmorphic features (DIDOD), DiGeorge syndrome (TBXI-associated), Dravet syndrome, Duane-radial raysyndrome, Ehlers-Danlos syndrome (classic-like), Ehlers-Danlos syndrome (vascular type), Feingold syndrome 1, Frontotemporal lobar degeneration with TDP43 inclusions (FTLD-TDP), GRN-related, GLUT I deficiency syndrome, Greig cephalopolysyndactyly syndrome, Hereditary hemorrhagic telangiectasia type 1, Holoprosencephaly 3, Holoprosencephaly 4, Holoprosencephaly 5, Holt-Oram syndrome, Hypoparathyroidism, sensorineural deafness, andrenal disease (HDR), Kleefstra syndrome 1, Klippel-Trenaunay syndrome (AAGF-related), Leri-Weill dyschondrosteosis, Marfan syndrome, Mental retardation and distinctive facial features with or without cardiac defects (MRFACD), Mental retardation, autosomal dominant 1, Mental retardation, autosomal dominant 19, Mental retardation, autosomal dominant 29, Nail-patella syndrome (NPS), Phelan-McDermid syndrome, Pitt-Hopkins syndrome, Primary pulmonary hypertension 1, Rett syndrome (congenital variant), Smith-Magenis syndrome (RAII associated), Sotos syndrome 1, Sotos syndrome 2, Stickler syndrome type I, Supravalvular aorticstenosis, SYNGAPI-related intellectual disability, Treacher Collins syndrome, Trichorhinophalangeal syndrome type I, Ulnar-mammary syndrome, van der Woude syndrome1, Waardenburg syndrome type 1, Waardenburg syndrome type 2A, and Waardenburg syndrometype 4C.

44. The method of any one of claims 41-43, wherein the subject is a mammal.

45. The method of claim 44, wherein the subject is a human.
